(19) United States
(12) Patent Application Publication
(10) Pub. No.: US 2023/0303504 A1
(43) Pub. Date: Sep. 28, 2023

(54) CRYSTALLINE SALTS OF TIZOXANIDE AND 2-HYDROXY-N-(5-CHLORO-1,3-THIAZOL-2-YL)BENZAMIDE (RM-4848) WITH ETHANOLAMINE, MORPHOLINE, PROPANOLAMINE, PIPERAZINE AND N-METHYLPIPERAZINE

(71) Applicant: Romark Laboratories, L.C., Tampa, FL (US)

(72) Inventors: Jean-Francois Rossignol, St. Petersburg, FL (US); Andrew V. Stachulski, Tampa, FL (US)

(73) Assignee: Romark Laboratories, L.C., Tampa, FL (US)

(21) Appl. No.: 18/017,038

(22) PCT Filed: Jul. 19, 2021
(86) PCT No.: PCT/US2021/042196

§ 371 Date: Jan. 19, 2023

Related U.S. Application Data
(60) Provisional application No. 63/054,072, filed on Jul. 20, 2020.

(51) Int. Cl. C07D 277/58; C07D 295/037; C07C 215/40

(52) U.S. Cl. CPC C07D 277/58; C07D 295/037; C07C 215/40

(57) ABSTRACT

The present invention refers to: crystalline tizoxanide amine salts, such as e.g. the ethanolamine salt, the morpholine salt, the propanolamine salt, the piperazine salt and the N-methylpiperazine salt, crystalline amine salts of 2-hydroxy-N-(5-halo-1,3-thiazol-2-yl) benzamide derivatives, such as e.g. the 2-hydroxy-N-(5-chloro-1,3-thiazol-2-yl)benzamide (RM-4848), a method for preparing a tizoxanide amine salt from its prodrug nitazoxanide (NTZ), pharmaceutical compositions comprising tizoxanide amine salts, tizoxanide amine salts for use as an antiviral or antiparasitic agent.

### RELATED APPLICATIONS

The present application is the U.S. National Stage of PCT/US2021/042196, filed Jul. 19, 2021, which claims priority to U.S. provisional patent application No. 63/054,072 filed Jul. 20, 2020, which is incorporated herein by reference in its entirety.

### FIELD

The present disclosure relates to thiazolide compounds and more specifically to salts of thiazolide compounds and their methods of making and use.

### SUMMARY

One embodiment is an amine containing salt of a compound having formula:

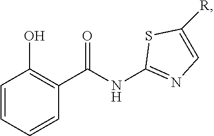

wherein R is NO₂ or a halogen.

Another embodiment is a pharmaceutical composition comprising a salt of tizoxanide and a pharmaceutically acceptable excipient, wherein when the composition is administered to a mammal, the composition provides a maximum concentration of tizoxanide in a plasma of a mammal in 1 hour or less.

Yet another embodiment is a pharmaceutical composition comprising a salt of tizoxanide and a pharmaceutically acceptable excipient, wherein when the composition is administered to a mammal, the composition provides a maximum concentration of tizoxanide in a plasma of the mammal faster than a pharmaceutical composition comprising nitazoxanide.

And yet another embodiment is a pharmaceutical composition comprising a salt of tizoxanide and a pharmaceutically acceptable excipient, wherein when the composition is administered to a mammal, the composition provides a AUC₀₋₁₂ₕ concentration of tizoxanide and glucorono tizoxanide in a plasma of the mammal of no less than that of a pharmaceutical composition comprising nitazoxanide.

And yet another embodiment is a method of making an amine containing salt of a thiazolide compound, comprising reacting a thiazolide compound of formula

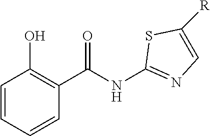

with an amine containing compound to produce an amine containing salt of the thiazolide compound, wherein R is NO₂ or Cl.

And yet another embodiment is an ethanolamine of tizoxanide.

### FIGURES

FIG. 1 is a plot showing median tizoxanide (T) concentrations (μg/mL) in plasma over 12 hours for RM-5071, RM-5072 and nitazoxanide (NTZ).

FIG. 2 is a plot showing median tizoxanide glucuronide (TG) concentrations (μg/mL) in plasma over 12 hours for RM-5071m,RM-5072 and NTZ.

FIG. 3 is a plot showing a sum of median free tizoxanide and glucuronidated tizoxanide concentrations (μg/mL) in plasma over 12 hours for RM-5071, RM-5072 and NTZ.

FIG. 4 shows examples of thiazolide amine containing salts.

FIG. 5 illustrates potential impurities in batches of RM-5071.

FIG. 6 shows scanning electron microscope (SEM) images of a batch of RM-5071 at X500 (top panel) and X1000 (bottom panel) magnifications.

FIG. 7 shows SEM images of a batch of desacetyl NTZ (tizoxanide) at X500 (top panel) and X1000 (bottom panel) magnifications.

FIG. 8 shows digital images (×10) of sample preparation for particle analysis pre-sonification, primary particles (LEFT) and post-sonification (RIGHT).

FIG. 9 shows overlays of particle measurement results for different applied pressures 0 bar (green), 1 bar (blue), 2 bar (purple=gray-blue), 4 bar (gray) and liquid dispersion average (red). Pressures 3 and 4 are overlayed.

FIG. 10 shows an overlay of liquid analysis (red) and 4-bar (green) and 1 bar (blue) with pre-dispersal and high energy venture analysis.

FIGS. 11A-B show thermal gravimetric analysis (TGA) thermograms of RM-5071 (A) and desacetyl-NTZA (B).

FIGS. 12A-B show differential scanning calorimetry (DSC) thermograms of RM-5071 (A) and desacetyl-NTZA (B).

FIG. 13 shows a titration curve of 1 mg/mL solution of RM-5071 with 0.01 N HCl.

FIG. 14 shows a titration curve of 1 mg/mL solution of RM-5071 with 0.02 N NaOH.

FIG. 15 presents UV-Vis absorption spectra for RM-5071 dissolved in Methanol.

FIG. 16 is absorbance (λmax=409 nm) of RM-5071 as a function of the solution concentration in water-supernatant.

FIG. 17 is H-NMR spectra for RM-5071 was acquired with NMR method (CH), with a 400 MHz equipment.

FIG. 18 is H-NMR spectra for RM-5071 corresponding to region of the spectra of the aromatic functionalities.

FIG. 19 is H-NMR spectra for RM-5071 corresponding to the aliphatic protons.

FIG. 20 shows Positive (top) and Negative (bottom) electrospray ionization mass spectrometry (ESI-MS) spectra for RM-5071.

FIG. 21 shows another Positive (top-blue) and Negative (bottom-black) ESI-MS spectra for RM-5071.

FIG. 22 is an overlay of Fourier Transform InfraRed (FTIR) spectra for RM-5071 (red) and desacetyl-NTZA (blue).

FIG. 23 schematically illustrates resonance structures for RM-5071.

FIG. 24 shows Attenuated Total Reflection Fourier Transform InfraRed (ATR-FT-IR) spectra for RM-5071 (red), desacetyl-NTZA (green) and a mixture of desacetyl-NTZA and ethanolamine 1:1 (black).

FIG. 25A-B show X-ray diffractograms of RM-5071 (A) and desacetyl-NTZA (B).

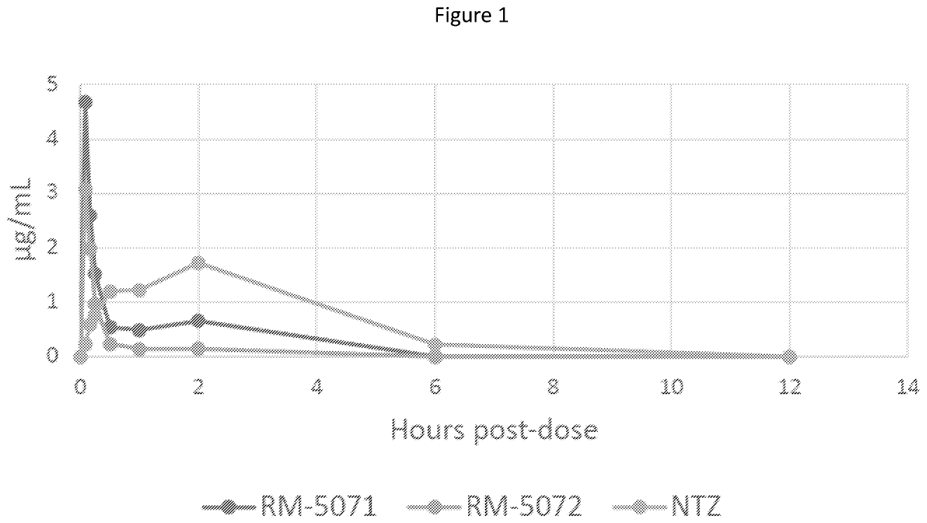

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. Other than in the operating examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims. In order that the present disclosure can be more readily understood, certain terms are first defined.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art are set forth throughout the detailed description.

“NMR” refers to nuclear magnetic resonance.

“Veq” refers to EQuivalence point Volume.

“AUC₀₋₁₂ₕ” refers to total area under the plasma concentration from time zero (i.e. from the time of administration) to 12 h after the administration.

Cₘₐₓ refers to a maximum plasma or serum concentration that a drug achieves after administration.

“FTIR” refers to Fourier Transform InfraRed spectroscopy.

“UV” refers to ultraviolet and visible spectroscopy.

“DMF” refers to dimethylformamide.

“DMA” refers to dimethylacetamide.

“PO” refer to per oral.

NTZ or NTZA refers to nitazoxanide, also known as 2-(acetolyloxy)-N-(5-nitro-2-thiazolyl) benzamide, which is a compound having the following structure:

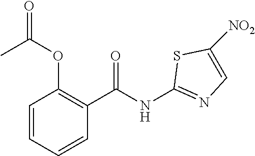 [SMILES: CC(=O)Oc1ccccc1C(=O)Nc1ncc([N+](=O)[O-])s1]

TIZ, desacetyl-NTZA, desacetyl-NTZ or desacetyl nitazoxanide refers to tizoxanide is the active circulating metabolite of nitazoxanide. Tizoxanide has the following formula:

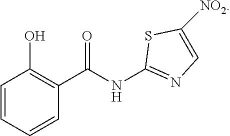 [SMILES: O=C(Nc1ncc([N+](=O)[O-])s1)c1ccccc1O]

Another metabolite of nitazoxanide is glucoronotizoxanide, which has the following formula:

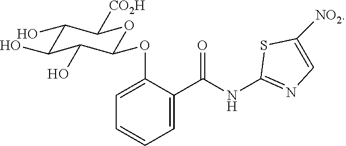 [SMILES: O=C(Nc1ncc([N+](=O)[O-])s1)c1ccccc1OC1OC(C(=O)O)C(O)C(O)C1O]

Nitazoxanide is approved in the United States for the treatment of diarrhea caused by Cryptosporidium parvum and Giardia lamblia.

RM-4848 is a substituted thiazolide having the same structure as tizoxanide, but having a chloro group substituted for the nitro group, thus resulting in the compound N-(5-chlorothiazol-2-yl)-2-hydroxybenzamide. RM-4848 has the following formula:

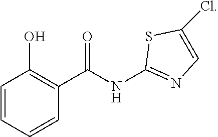 [SMILES: O=C(Nc1ncc(Cl)s1)c1ccccc1O]

Thiazolide compounds may be synthesized, for example, according to published procedures U.S. Pat. Nos. 3,950,351 and 6,020,353, PCTWO2006042195A1 and US2009/0036467A.

Pharmaceutical compositions containing nitazoxanide and its metabolite, tizoxanide, were originally developed and marketed for treating intestinal parasitic infections. Various applications of nitazoxanide, tizoxanide and other thiazolide compounds, such as RM-4848. are disclosed, for example, U.S. Patent Nos. RE47,786, U.S. Pat. Nos. 10,383,855, 10,363,243, 10,358,428, 10,336,058, RE47,404, U.S. Pat. No. 10,100,023, RE46,724, U.S. Pat. Nos. 9,827,227, 9,820,975, 9,351,937, 9,345,690, 9,126,992, 9,107,913, 9,023,877, 8,895,752, 8,846,727, 8,772,502, 8,633,230,8,524,278, 8,124,632, 7,645,783, 7,550,493, 7,285,567, 6,117,894, 6,020,353, 5,968,961, 5,965,590, 5,935,591, 5,886,013, 5,859,038, 5,856,348 as well as in U.S. patent application publications Nos. 20200038377, 20190321338, 20190307730, 20190291404, 20190276417, 20190040026, 20180126722, 20180085353, 2018078533, 20170334868, 20170281603, 20160243087, 20160228415, 2015025768, 20140341850, 20140112888, 20140065215, 20120294831, 20120122939, 20120108592, 20120108591, 20100330173, 20100292274, 20100209505, 20090036467, 20080097106, 20080097106, 20080096941, 20070167504, 20070015803, 20060194853, 20060089396, 20050171169, each of which is incorporated herein by reference in its entirety.

The present inventors developed novel salts of a thiazolide compound of the following formula:

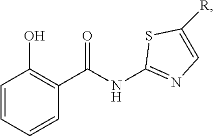

where R is NO₂ or a halogen, such as Cl or Br.

In some embodiments, a salt of the thiazolide compound may be an amine containing salt. As used herein, the term amine containing salt refers to a salt, which has a counterion, which contains one or more amine groups, such as primary amine groups, secondary amine groups or tertiary amine groups.

In some embodiments, the amine containing salt may be an alkyl amine salt, an oxaakyl amine salt or a cycloalkyl amine salt.

As used herein, “alkyl amine” may be an alkyl group having one or more amine groups, such as primary amine groups, secondary amine groups or tertiary amine groups.

As used herein, the term “alkyl,” as used herein, alone or in combination, refers to a straight-chain or branched-chain alkyl radical containing 1 to 10, 1 to 6, or 1 to 4 carbon atoms. The term “alkyl groups” may be used in its broadest sense. Alkyl groups may be optionally substituted. Examples of alkyl radicals include methyl, ethyl, n-propyl, isopropyl, n-butyl, isobutyl, sec-butyl, tert-butyl, pentyl, iso-amyl, hexyl.

In some embodiments, alkyl amine may be an alkyl containing one or more terminal amino-group. Examples of such alkyl amines include methyl amine, ethyl amine, n-propyl amine, n-butylamine, sec-butylamine, tert-butylamine, and isobutylamine.

In some embodiments, alkyl amine may be an alkyl having one or more CH₂ groups substituted with NH.

As used herein, oxaalkyl refers to an alkyl having one or more CH₂ substituted with O and/or having CH₃ group replaces with OH.

In some embodiments, an oxaalkyl amine may be an alkyl having a terminal OH group and a terminal amino group. Examples of such amines include ethanol amine, propanolamine, n-butanolamine

The term “cycloalkyl,” as used herein, alone or in combination, refers to a saturated monocyclic radical wherein each cyclic moiety contains from 3 to 12, from 3 to 8 or from 3 to 6 carbon atom ring members and which may optionally be optionally substituted. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like.

Cycloalkyl amine refers to a cycloalkyl having one or more CH₂ groups substituted with NH. In some embodiments, cycloalkyl amine may a cycloalkyl having one CH₂ group substituted with NH. Yet in some embodiments, cycloalkyl amine may a cycloalkyl having more than one, i.e. two or more CH₂ groups substituted with NH.

In some embodiments, one or more CH₂ groups in cycloalkyl amine may be further substituted with O or CH₃N.

Examples of cycloalkyl amines include morpholine and N-methylpiperazine.

In some embodiments, the amine containing salt may be an ethanolamine salt, a morpholine salt, a propanol amine salt or N-methylpiperazine salt.

In some embodiments, the amine containing salt of the thiazolide compound may a salt of a liquid amine containing base, such as ammonia, methylamine, diethylamine, ethanolamine, dicyclohexylamine, N-methylmorpholine, ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine.

In some embodiments, the amine containing salt may be a crystalline salt.

In some embodiments, the amine containing salt may be a pure salt having a purity of at least 90% or at least 92% or at least 94% or at least 95% or at least 96% or at least 97% or at least 98% or at least 98.5% or at least 99% or at least 99.1% or at least 99.2% or at least 99.3% or at least 99.4% or at least 99.5%. The pure amine containing salt may be in a batch containing at least 10 g or at least 20 g or at least 30 g or at least 40 g or at least 50 g or at least 60 g or at least 70 g or at least 80 g or at least 90 g or at least 100g or at least 150 g or at least 200 g or at least 250 g or at least 300 g or at least 350 g or at least 400 g or at least 450 g or at least 500 g or at least 600 g or at least 700 g or at least 800 g or at least 900 g or at least 1000 g or at least 1200 g or at least 1400 g or at least 1500 g of the salt or at least 2000 g of the salt or at least 4000 g of the salt or at least 5000 g of the salt or at least 8000 g of the salt or at least 10000 g of the salt or at least 15000 g of the salt or at least 20000 g of the salt or at least 25000 g of the salt or at least 30000 g of the salt or at least 35000 g of the salt or at least 40000 g of the salt.

In some embodiments, a salt of the thiazolide compound, such as an amine containing salt of the thiazolide compound, may be administered as a part of a pharmaceutical composition. The pharmaceutical composition may include in addition to the salt of the thiazolide compound may include a carrier, such as a pharmaceutically acceptable carrier. The term “carrier” may be used in its broadest sense. For example, the term “carrier” refers to any carriers, diluents, excipients, wetting agents, buffering agents, suspending agents, lubricating agents, adjuvants, vehicles, delivery systems, emulsifiers, disintegrants, absorbents, preservatives, surfactants, colorants, flavorants, and sweeteners. In some embodiments, the carrier may be a pharmaceutically acceptable carrier, a term narrower than carrier, because the term pharmaceutically acceptable carrier” means a non-toxic that would be suitable for use in a pharmaceutical composition. Actual dosage levels of active ingredients in the pharmaceutical compositions may vary so as to administer an amount of the active compound(s) that is effective to achieve the desired therapeutic response for a particular patient.

The selected dose level may depend on the activity of the thiazolide compound, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient may depend on a variety of factors, including the body weight, general health, diet, time and route of administration and combination with other therapeutic agents and the severity of the condition or disease being treated.

The pharmaceutical compositions may be administered systemically, for example, in an oral formulation, such as a solid oral formulation. For example, it may be in the physical form of a powder, tablet, capsule, lozenge, gel, solution, suspension, syrup, or the like. In some embodiments, the pharmaceutical composition may be in a form of a formulation disclosed in U.S. Pat. Nos. 8,524,278 and 9,351,937. Such formulation may, for example, include a controlled release portion and an immediate release portion, such that at least one of the controlled release portion and the immediate release portion includes a salt of a thiazolide compound, such an amine containing salt of the thiazolide compound. For example, in some embodiments, the controlled release portion may include a salt of a thiazolide compound, such an amine containing salt of the thiazolide compound, while the immediate release portion may include a salt of the thiazolide compound, which may be the same or different from the salt in the controlled release portion, and/or the thiazolide compound per se. Yet in some embodiments, the immediate release portion may include a salt of a thiazolide compound, such an amine containing salt of the thiazolide compound, while the controlled release portion may include a salt of the thiazolide compound, which may be the same or different from the salt in the immediate release portion, and/or the thiazolide compound per se. These compositions may be administered in a single dose or in multiple doses which are administered at different times.

In some embodiments, the total amount of the thiazolide compound, in the composition containing a salt of the thiazolide compound, such as an amine-containing salt of the thiazolide compound, may be from about 20% to about 95% or from about 30% to about 90% or from about 35% to about 85% or from about 60% to about 75% by weight of the composition. The composition may be formulated for immediate release, controlled release or sustained release. The compositions may contain one or more additional pharmaceutically acceptable additives or excipients. These excipients are therapeutically inert ingredients that are well known and appreciated in the art. As used herein, the term “inert ingredient” may refer to those therapeutically inert ingredients that are well known in the art of pharmaceutical manufacturing, which can be used singly or in various combinations, and include, for example, diluents, disintegrants, binders, suspending agents, glidants, lubricants, fillers, coating agents, solubilizing agent, sweetening agents, coloring agents, flavoring agents, and antioxidants. See, for example, Remington: The Science and Practice of Pharmacy 1995, edited by E. W. Martin, Mack Publishing Company, 19th edition, Easton, Pa.

Examples of diluents or fillers include, but are not limited to, starch, lactose, xylitol, sorbitol, confectioner's sugar, compressible sugar, dextrates, dextrin, dextrose, fructose, lactitol, mannitol, sucrose, talc, microcrystalline cellulose, calcium carbonate, calcium phosphate dibasic or tribasic, dicalcium phosphaste dehydrate, calcium sulfate, and the like. The amount of diluents or fillers may be in a range between about 2% to about 15% by weight of the entire composition.

Examples of disintegrants include, but are not limited to, alginic acid, methacrylic acid DVB, cross-linked PVP, microcrystalline cellulose, sodium croscarmellose, crospovidone, polacrilin potassium, sodium starch glycolate, starch, including corn or maize starch, pregelatinized starch and the like. Disintegrant(s) typically represent about 2% to about 15% by weight of the entire composition.

Examples of binders include, but are not limited to, starches such as potato starch, wheat starch, corn starch; microcrystalline cellulose; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, and the like. The amount of binder(s) is about 0.2% to about 14% by weight of the entire composition.

Examples of glidants include, but are not limited to, silicon dioxide, colloidal anhydrous silica, magnesium trisilicate, tribasic calcium phosphate, calcium silicate, magnesium silicate, colloidal silicon dioxide, powdered cellulose, starch, talc, and the like. The amount of glidant(s) is about 0.01% to about 0.3% by weight of the entire composition.

Examples of lubricants include, but are not limited to, magnesium stearate, aluminum stearate, calcium stearate, zinc stearate, stearic acid, polyethylene glycol, glyceryl behenate, mineral oil, sodium stearyl fumarate, talc, hydrogenated vegetable oil and the like. The amount of lubricant(s) is about 0.2% to about 1.0% by weight of the entire composition.

The compositions may contain a binder that is a low-viscosity polymer. Examples of low-viscosity polymers include, but are not limited to, low-viscosity hydroxypropyl methylcellulose polymers such as those sold by Dow Chemical under the tradename “MethoceL™” (e.g., Methocel E5OLV™, Methocel K100LVR™, and Methocel F50LVR™) and low-viscosity hydroxyethylcellulose polymers. The low-viscosity polymer is typically present at about 10% to about 20%, or about 10% to about 15%, or preferably about 12%, of the total weight of the entire composition, or, in those embodiments having controlled release and immediate release portions, the low-viscosity polymer in the controlled release portion is typically present at about 15% to about 20%, preferably about 18%, of the weight of the controlled release portion.

The compositions may further comprise a coating material. The coating material is typically present as an outer layer on the dosage form that completely covers the formulation. For example, in some embodiments, the dosage form is an oral tablet in which the controlled release portion forms a first layer of the tablet and the immediate release portion forms a second layer that is deposited on top of the first layer to form a core tablet. In such embodiments, e.g., the coating material can be in the form of an outer coating layer that is deposited on top of the core tablet. The coating material typically is about 1% to about 5% by weight of the composition, and may comprise hydroxypropylmethylcellulose and/or polyethylene glycol, and one or more excipients selected from the group comprising coating agents, opacifiers, taste-masking agents, fillers, polishing agents, coloring agents, antitacking agents and the like. Examples of film-coating substances and methods for using such coating substances are well known to those of skill in the art.

In some embodiments, a salt of the thiazolide compound or a pharmaceutical composition comprising such salt, when administered to a mammal, such as a human being, may provide a maximum concentration of the compound in a plasma of the mammal in 2 hours or less or in 1.5 hours or less or in 1 hour or less or in 50 min or less or in 40 minutes or less or in 30 minutes or less or in 25 minutes or less or in 20 minutes or less or in 15 minutes or less or in 10 minutes or less or in 5 minutes or less after the administering. For example, a salt of tizoxanide, such as an amine containing salt of tizoxanide, which may be, for example, an ethanolamine salt of tizoxanide or a morpholine salt of tizoxanide, or a pharmaceutical composition comprising such salt when administered to a mammal, such as a human being, may provide a maximum concentration of tizoxanide in a plasma of the mammal in 2 hours or less or in 1.5 hours or less or in 1 hour or less or in 50 min or less or in 40 minutes or less or in 30 minutes or less or in 25 minutes or less or in 20 minutes or less or in 15 minutes or less or in 10 minutes or less or in 5 minutes or less after the administering. In some embodiments, a salt of tizoxanide, such as an amine containing salt of tizoxanide, which may be, for example, an ethanolamine salt of tizoxanide or a morpholine salt of tizoxanide, or a pharmaceutical composition comprising such salt when orally administered to a mammal, such as a human being, may provide a maximum concentration of tizoxanide in a plasma of the mammal in 2 hours or less or in 1.5 hours or less or in 1 hour or less or in 50 min or less or in 40 minutes or less or in 30 minutes or less or in 25 minutes or less or in 20 minutes or less or in 15 minutes or less or in 10 minutes or less or in 5 minutes or less after the administering.

In some embodiments, a salt of tizoxanide, such as an amine containing salt of tizoxanide, which may be, for example, an ethanolamine salt of tizoxanide or a morpholine salt of tizoxanide, when administered to a mammal, such as a human being, may provide a maximum concentration of tizoxanide in a plasma of the mammal faster than nitazoxanide or an otherwise identical pharmaceutical composition comprising nitazoxanide instead of the salt of tizoxanide. In some embodiments, a salt of tizoxanide, such as an amine containing salt of tizoxanide, which may be, for example, an ethanolamine salt of tizoxanide or a morpholine salt of tizoxanide, when orally administered to a mammal, such as a human being, may provide a maximum concentration of tizoxanide in a plasma of the mammal faster than nitazoxanide or an otherwise identical pharmaceutical composition comprising nitazoxanide instead of the salt of tizoxanide.

In some embodiments, a salt of tizoxanide, such as an amine containing salt of tizoxanide, which may be, for example, an ethanolamine salt of tizoxanide, when administered to a mammal, such as a human being, may provide a AUC₀₋₁₂ₕ concentration of tizoxanide and glucorono tizoxanide in a plasma of the mammal of no less than that of nitazoxanide or an otherwise identical pharmaceutical composition comprising nitazoxanide instead of the salt of tizoxanide. In some embodiments, a salt of tizoxanide, such as an amine containing salt of tizoxanide, which may be for example an ethanolamine salt of tizoxanide, when orally administered to a mammal, such as a human being, may provide a AUC₀₋₁₂ₕ concentration of tizoxanide and glucorono tizoxanide in a plasma of the mammal of no less than that of nitazoxanide or an otherwise identical pharmaceutical composition comprising nitazoxanide instead of the salt of tizoxanide.

An amine containing salt of a thiazolide compound may be prepared by reacting a thiazolide compound of formula

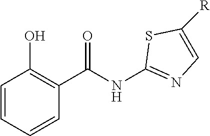

with an amine containing compound, which may be a liquid amine containing compound, to produce an amine containing salt of the thiazolide compound, where R is NO₂ or Cl.

For such reacting, the thiazolide compound, such as tizoxanide may be dispersed in a solvent, which may be for example an alcohol, such as methanol or ethanol. An amine containing compound, which may be a liquid amine containing compound, such as ethanolamine, propanolamine, morpholine, or N-methylpiperazine, may be added to the dispersion. In some embodiments, a temperature of the mixture may be kept below 30° C. or below 25° C. In some embodiments, the mixture may be stirred. A reaction time may vary. In some embodiments, the reaction time may be from 30 minutes to 4 hours, or from 1 hour to 3 hours, such as about 2 hours. The mixture may be filtered and the product containing the amine containing salt of the thiazolide compound may be washed using a solvent, which may include an alcohol, such as methanol or ethanol, and/or an acetic acid ester, such as ethyl acetate. The product containing the amine containing salt of the thiazolide compound may be dried using one or more of vacuum, which may be a pressure below about 100 mbar, e.g. 0.2 to 50 mbar or 0.5 to 20 mbar or 1 to 10 mbar, and an elevated temperature, which may be from 50C to 80C or from 50C to 70C or from 55C to 65C or any subrange or value within these ranges. In some embodiments, the product containing the amine containing salt of the thiazolide compound may be dried under vacuum at a temperature between about 15C to about 30 C, such as 20C. The dried solid product of the amine containing salt of the thiazolide compound may be milled and/or crushed.

In some embodiments, when a produced batch of the amine containing salt contains an excess of the amine containing compound with respect to the thiazolide compound, the batch may be purified to get rid of the excess of amine containing compound. Such purification may be performed by reslurry of the product in a solvent which may include an alcohol, such as methanol or ethanol, and/or water. The excess of the amine containing compound with respect to the thiazolide compound may be determined by measuring a molar ratio of the amine containing compound and the thiazolide compound in the batch by a quantitative technique such as HPLC or LC-MS.

For example, when a produced batch of an ethanolamine salt of tizoxanide contains an excess of ethanolamine with respect to tizoxanide, the batch may be purified to get rid of the excess of the ethanolamine. The excess of the ethanolamine with respect to the tizoxanide in the batch may be determined by measuring a molar ratio of the ethanolamine and the tizoxanide in the produced batch by a quantitative technique such as HPLC or LC-MS. For example, the batch may have an excess of the ethanolamine with respect to the tizoxanide if a molar ratio between the ethanolamine and the tizoxanide is greater than 1.00 or greater than 1.05. If the excess is determined, then after the purification, the purified batch may have a molar ratio between the ethanolamine and the tizoxanide from 0.9 to 1.00 or from 0.95 to 1.00 or from 0.96 to 1.00 or from 0.97 to 1.00 or from 0.98 to 1.00 or from 0.99 to 1.00.

The thiazolide compound, such as tizoxanide, may be produced from its respective prodrug. For example, tizoxanide may prepared from nitazoxanide by heating a solution comprising nitazoxanide to a first elevated temperature, such as at least 50° C., or at least 55° C. or at least 60° C. or at least 65° C. or at least 70° C. or at least 75° C. A solvent in the solution may be a polar solvent, such as, for example, dimethylacetamide or dimethylformamide. In some embodiments, the nitazoxanide may be dispersed in the solvent to form the solution. In some embodiments, an acid, such as HCl, which may be a dilute acid, such as HCl at about 0.5 M to 3 M, such as 1 M, may be added to the solution. After the acid addition, in some embodiments, temperature of the mixture may be further elevated to a second elevated temperature, such as at least 65° C. or at least 70° C. or at least 75° C. In some embodiments, each of the first and the second elevated temperatures may be no greater than 100° C. or no greater than 95° C. or no greater than 90° C. or no greater than 85° C. or no greater than 80° C. The heating at the second elevated temperature may last for at least 10 hours, at least 15 hours or at least 20 hours or at least 25 hours or at least 30 hours. In some embodiments, the heating at the second elevated temperature may last from 10 hours to 70 hours or from 15 hours to 65 hours or from 20 hours to 60 hours or from 30 hours to 50 hours or any value or subrange within these ranges. After a conversion of nitazoxanide to tizoxanide, the solution may be cooled down, for example, to room temperature, such as around 25° C., and neutralized with a base, e.g. KOH or NaOH, such as about 1 M NaOH. During the neutralization, the temperature may be kept below 30° C. or below 25° C. The tizoxanide formed from nitazoxanide may be used for a subsequent salt formation without drying.

In some embodiments, conversion of nitazoxanide into tizoxanide may be performed without using concentrated acid and/or concentrated base; without handling strong acidic or alkaline mixture based on any parameter); and/or without using volatile solvents, such as solvents having a boiling temperature below 100° C.

In some embodiments, tizoxanide may be prepared from nitazoxanide by preparing an aqueous solution of ammonia in tetrahydrofuran, followed by evaporation, suspension in a cold acid, such as cold aqueous HCl, and filtering.

Methods of converting nitazoxanide into tizoxanide are also disclosed, for example, in Rossignol and Stachulski, J. Chem. Res. (S), 1999, 44-45, which is incorporated herein by reference in its entirety.

A salt of a thiazolide compound, such as tizoxanide or RM-4848, and a pharmaceutical compositions comprising such a salt may be used for one or more of the same purposes for which nitazoxanide, tizoxanide and/or RM-4848 are known to be useful. For example, the salt or the pharmaceutical composition may be used for administering to a subject, such as a human being, for treating a disease or disorder, which may be treated with nitazoxanide or tizoxanide, such as an influenza infection, an influenza-like illness, a respiratory infection, a disease or condition caused by a virus belonging to the genus Enterovirus, such as rhinovirus and/or enterovirus, a disease or condition caused by a virus belonging to the Coronaviridae family, such as a coronavirus, a disease or condition caused by a virus belonging to the Paramyxoviridae family, such as respiratory syncytial virus, Sendai virus or Hendra virus, hepatitis C, hepatitis B, including chronic hepatitis B, intestinal parasitic infections, diarrhea caused by Cryptosporidium parvum and Giardia lamblia. For therapeutic purposes, salt of a thizolide compound, such as tizoxanide, may be administered to a subject, such a human being, in a therapeutically effective amount, which may be an amount of the disease, which is sufficient to ameliorate one or more symptoms of a disease or disorder, which may be treated with nitazoxanide and/or tizoxanide.

In some embodiments, the amine containing salt of tizoxanide may be an ethanolamine salt of tizoxanide. In some embodiments, such salt may be in a form of particles having an average size of no greater than 50 microns or no greater than 45 microns or no greater than 40 microns or no greater than 30 microns or no greater than 25 microns or no greater than 20 microns. In some embodiments, the ethanolamine salt of tizoxanide may contain fine particles, such that at least 50% or at least 60% or at least 70% or at least 80% or at least 90% of the particles have a size from about 1 micron to about 60 microns or from about 2 microns to about 50 microns or from about 3 microns to about 45 microns or from about 4 microns to about 40 microns or from about 4 microns to about 35 microns or from about 4 microns to about 30 microns or any value or subrange within these ranges. In some embodiments, the ethanolamine salt of tizoxanide may further optionally contain no more than 30% or no more than 20% or no more than 10% of coarse particles having a size of at least about 100 microns, such as from about 100 microns to about 2000 microns or from about 100 microns to about 1500 microns or from about 100 microns to about 1000 microns. In some embodiments, a batch of the ethanolamine salt of tizoxanide may be prepared from a batch of tizoxanide, so that the batch of the ethanolamine salt of tizoxanide has an average particle size and/or particle distributing distinct from that of the batch of tizoxanide.

In some embodiments, the ethanolamine salt of tizoxanide may have a melting temperature from about 144C to about 150C or from about 146C to about 148C.

In some embodiments, the ethanolamine salt of tizoxanide may be in a crystalline form.

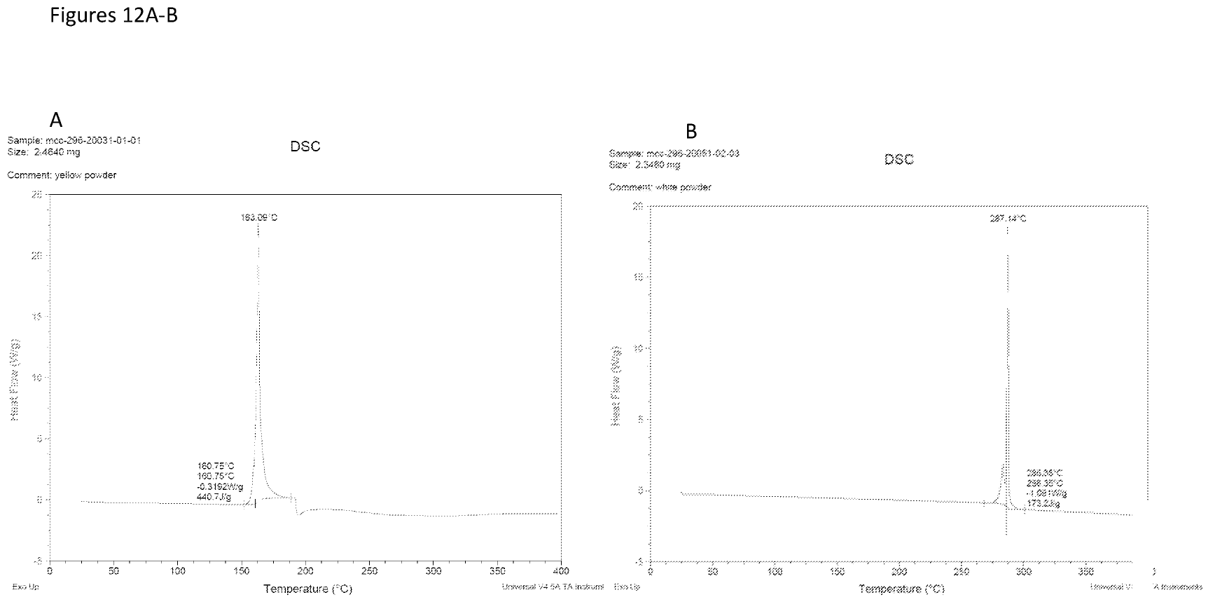

In some embodiments, the ethanolamine salt of tizoxanide may have a differential scanning calorimetry (DSC) curve as in FIG. 12A.

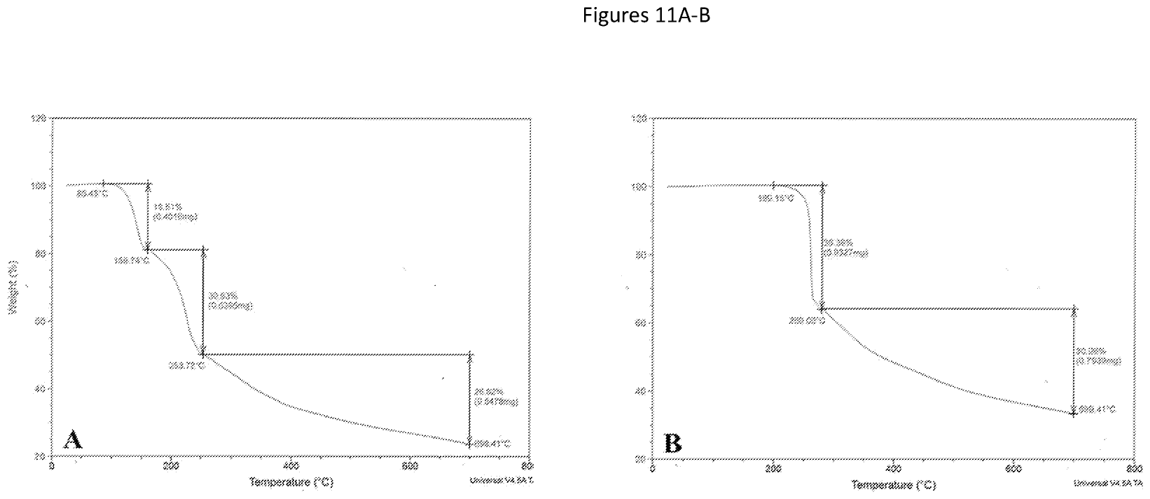

In some embodiments, the ethanolamine salt of tizoxanide may have a thermal gravimetric analysis (TGA) thermogram as in FIG. 11A.

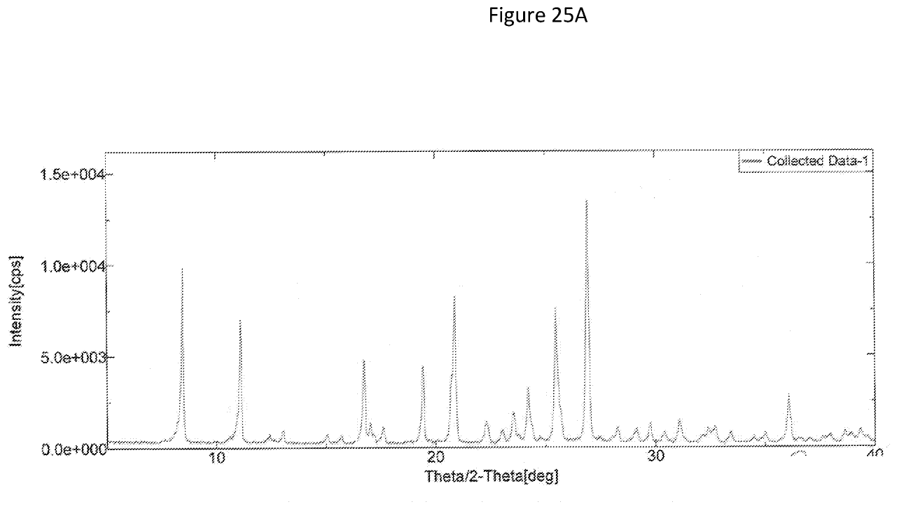

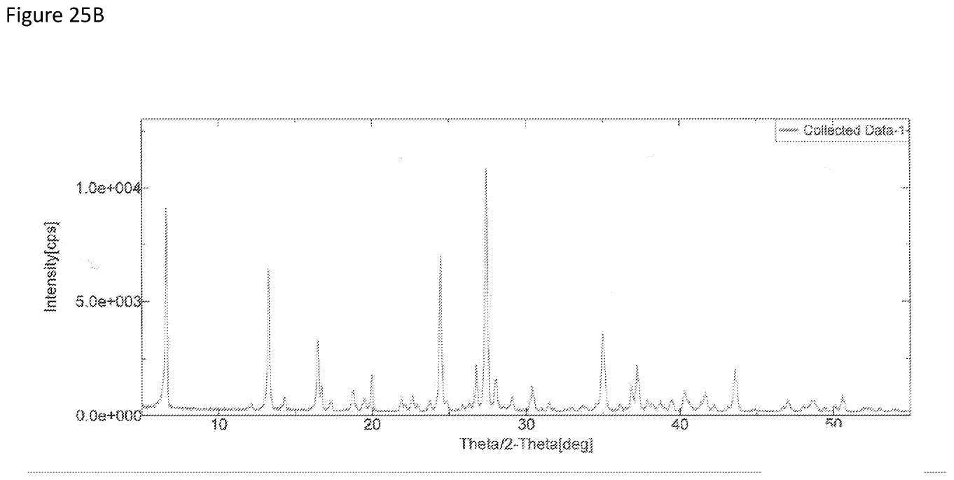

In some embodiments, the ethanolamine salt of tizoxanide may have an X-ray powder diffractogram as determined on a diffractometer using Cu-Kβ radiation at a wavelength of 1.39222 Å as in FIG. 25A.

In some embodiments, the ethanolamine salt of tizoxanide may have an X-ray powder diffractogram as determined on a diffractometer using Cu-Kβ radiation at a wavelength of 1.39222 Å, such that the diffractogram has one or more peaks at about 8.5°, about 11.2°, about 16.8°, about 19.5°, about 20.9°, about 25.6°, about 27.0° and about 36.1° 2θ.

In some embodiments, the ethanolamine salt of tizoxanide may have an X-ray powder diffractogram as determined on a diffractometer using Cu-Kβ radiation at a wavelength of 1.39222 Å, such that the diffractogram has one or more peaks at 8.5° ±0.2°, 11.2° ±0.2° , 16.8° ±0.2°, 19.5° ±0.2°, 20.9° ±0.2°, 25.6° ±0.2°, 27.0° ±0.2°, and 36.1° ±0.2° 2θ.

In some embodiments, the ethanolamine salt of tizoxanide may have an X-ray powder diffractogram as determined on a diffractometer using Cu-Kβ radiation at a wavelength of 1.39222 Å, such that the diffractogram has peaks at 8.5° ±0.2°, 11.2° ±0.2°, 16.8° ±0.2° , 19.5° ±0.2°, 20.9° ±0.2°, 25.6° ±0.2°, 27.0° ±0.2°, and 36.1° ±0.2° 2θ.

In some embodiments, the ethanolamine salt of tizoxanide may be in a form of a batch. Such batch may contain at least 0.1 kg, or at least 0.2 kg or at least 0.3 kg or at least 0.4 kg or at least 0.5 kg or at least 0.6 kg or at least 0.7 kg or at least 0.8 kg or at least 0.9 kg or at least 1.0 kg or at least 1.2kg or at least 1.5 kg or at least 2.0 kg or at least 2.3 kg or at least 2.5 kg or at least 3.0 kg or at least 4 kg or at least 5 kg or at least 7 kg or at least 10 kg or at least 15 kg or at least 20 kg or at least 25 kg or at least 30 kg or at least 35 kg or at least 40 kg of the ethanolamine salt of tizoxanide. In some embodiments, a molar ratio between ethanolamine and tizoxanide in such batches may be between 0.9 and 1.00 or between 0.95 and 1.00 or from 0.96 to 1.00 or from 0.97 to 1.00 or from 0.98 to 1.00 or from 0.99 to 1.00. A molar ratio between ethanolamine and tizoxanide in a bacth of the ethanolamine salt may be determined by a number of techniques, including high performance liquid chromatography (HPLC) and liquid chromatography-mass spectrometry (LC-MS).

In some embodiments, an ethanolamine salt of tizoxanide may be prepared by reacting tizoxanide with ethanolamine. Tizoxanide for reacting with the ethanolamine may be previously prepared from nitazoxanide. Thus, in some embodiments, the ethanolamine salt of tizoxanide may be prepared via a process, which may involve two steps: step 1: preparation of tizoxanide from nitazoxanide and step 2: preparation of the ethanolamine salt of tizoxanide from the tizoxanide produced in step 1.

Embodiments described herein are further illustrated by, though in no way limited to, the following working examples.

### EXAMPLES

### Example 1. 2-Hydroxybenzoyl-N-[(5-Nitro)Thiazol-2-yl]Amide, Ethanolamine Salt. (RM-5071)

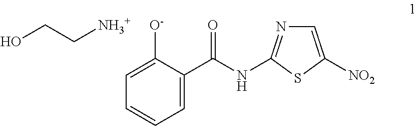

Tizoxanide (sc. 2-Hydroxybenzoyl-N-[(5-nitro)thiazol-2-yl]amide, 0.53 g, 2 mmol) was suspended in methanol (MeOH, 20 ml) containing ethanolamine (0.15 mL). The suspension was warmed to 50° C. for a few minutes, giving a virtually clear yellow solution which was filtered and concentrated to 5 mL when crystallization readily began. Diethyl ether (Et₂O, 5 mL) was added and the mixture was cooled to 0° C. to complete crystallization. Filtration, washing with Et₂O containing a little MeOH, afforded the title salt 1 (0.49 g, 75%) as a yellow crystalline solid; Melting point: 158-160° C. (decomposition); Found: C, 44.1; H, 4.2; N, 17.35; S, 9.8. C₁₂H₁₄N₄O₅S requires C, 44.2; H, 4.3; N, 17.2; S, 9.8%; 1H NMR [400 MHz, (CD₃)₂SO]δ2.86 (2H, t, CH₂CH₂), 3.57 (2H, t, CH₂CH₂), 5.20 (1H, br s, OH), 6.81 (2H, m, ArH), 7.32 (1H, m, ArH), 7.67 (3H, br s, NH₃⁺), 7.91 (1H, m, ArH), 8.51 (1H, s, 4′-H) and 14.71 (1H, br s, NH); 13C NMR [100 MHz, (CD₃)₂SO]δ41.6, 57.9, 117.5, 118.2, 119.9, 130.1, 133.4, 137.9, 145.9, 161.3, 171.6 and 172.2; m/z (-ve ion electrospray mode) 264 [(M-H)⁻]. Found: m/z, 264.0092. C₁₀H₆N₃O₄S requires m/z, 264.0085.

### Example 2. 2-Hydroxybenzoyl-N-[(5-Chloro)Thiazol-2-yl]Amide, Ethanolamine Salt 2

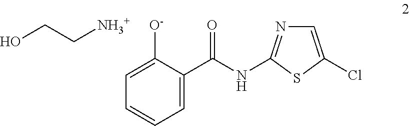

This salt was prepared similarly to the salt of Example 1 using R1V14848, viz. 2-hydroxybenzoyl-N-[(5-chloro)thiazol-2-yl]amide (0.51 g, 2 mmol), giving the product 2 (0.48 g, 76%); Found: C, 45.7; H, 4.5; N, 13.35; S, 10.15. C₁₂H₁₄ClN₃O₃S requires C, 45.6; H, 4.5; N, 13.3; S, 10.15%; 1H NMR [400 MHz, (CD₃)₂SO]δ2.86 (2 H, t, CH₂CH₂), 3.58 (2H, t, CH₂CH₂), 5.20 (1 H, br s, OH), 6.67-6.73 (2 H, 2m, ArH), 7.20 (1 H, m, ArH), 7.23 (1 H, s, 4′-H) and 7.83 (1 H, dd, ArH); the NH₃⁺ appears as a very broad signal centred at δ7.65; 13C NMR [100 MHz, (CD₃)₂SO]δ41.7, 58.0, 114.3, 116.8, 117.4, 120.4, 129.4, 132.1, 135.3, 162.4, 164.5 and 169.5; m/z (-ye ion electrospray mode) 253 [(M-H)⁻]. Found: m/z, 252.9849. C₁₀H₆³⁵ClN₂O₂S requires m/z, 252.9844.

### Example 3. 2-Hydroxybenzoyl-N-[(5-Nitro)Thiazol-2-yl]Amide, Morpholine Salt 3 (RM-5072)

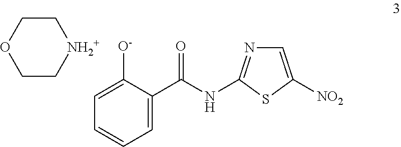 [SMILES: C1COCC[NH2+]1 ; O=C(Nc1ncc([N+](=O)[O-])s1)c1ccccc1[O-]]

This salt was prepared similarly to the salt of example 1 from tizoxanide (2-hydroxybenzoyl-N-[(5-nitro)thiazol-2-yl]amide; 0.53 g, 2 mmol) and morpholine (0.24 mL), giving 3 as a yellow microcrystalline solid (0.66 g, 94%); Found: C, 47.7; H, 4.6; N, 16.0; S, 9.2. C₁₄H₁₆N4O₅S requires C, 47.7; H, 4.6; N, 15.9; S, 9.1%; 1H NMR [400 MHz, (CD₃)₂SO]δ3.11, 3.75 (8 H, 2m, 2xCH₂CH₂), 6.82 (2 H, m, ArH), 7.31 (1 H, t, ArH), 7.91 (1 H, m, ArH) and 8.51 (1 H, s, 4′-H); 13C NMR [100 MHz, (CD₃)₂SO]δ43.4, 63.8, 117.5, 118.2, 119.8, 130.1, 133.4, 138.0, 145.9, 161.3, 171.5 and 172.1.

### Example 4. 2-Hydroxybenzoyl-N-[(5-Chloro)Thiazol-2-yl]Amide, Morpholine Salt 4

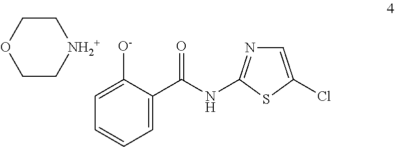 [SMILES: C1COCC[NH2+]1 ; O=C(Nc1ncc(Cl)s1)c1ccccc1[O-]]

This salt was prepared similarly to the salt of Example 1 from RM4848 (2-hydroxybenzoyl-N-[(5-chloro)thiazol-2-yl]amide; 0.51 g, 2 mmol) and morpholine (0.24 mL). In this case, the first solid which separated was unchanged RM4848; concentration of the mother liquors afforded the desired salt 4 (0.198 g, 29%); Found: C, 49.4; H, 4.9; N, 12.2; S, 9.2.

C₁₄H₁₆ClN₃O₃S requires C, 49.2; H, 4.7; N, 12.3; S, 9.4%; 1H NMR [400 MHz, (CD₃)₂SO]δ3.04 (4 H, m), 3.71 (4 H, m), 6.70 (2 H, 2m, ArH), 7.21 (1 H, t, ArH), 7.24 (1 H, s, 4′-H) and 7.83 (1 H, d, ArH); 13C NMR [100 MHz, (CD₃)₂SO]δ43.9, 64.5, 114.4, 116.9, 117.4, 120.3, 129.5, 132.2, 135.3, 162.2, 164.2 and 169.3.

### Example 5. 2-Hydroxybenzoyl-N-[(5-Nitro)Thiazol-2-yl]Amide, 3-Amino-1-Propanol Salt

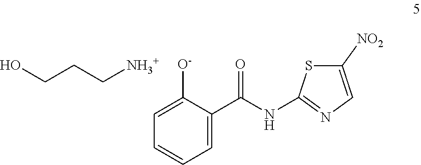 [SMILES: [NH3+]CCCO ; O=C(Nc1ncc([N+](=O)[O-])s1)c1ccccc1[O-]]

This was prepared similarly to 1 from tizoxanide (0.53 g, 2 mmol) and 3-amino-1-propanol (0.19 mL) giving the desired salt 5 as orange crystals (0.395 g, 58%). Found: C, 5.9; H, 4.7; N, 16.4: S, 9.5. C₁₃H₁₆N₄O₅S requires C, 45.9; H, 4.7; N, 16.5; S, 9.4%; 1H NMR [400 MHz, (CD₃)₂SO] δ_{H} 1.68 (2 H, m, CH₂CH₂CH₂), 2.86 (2 H, t, CH₂CH₂), 3.30 (1 H, br s, OH), 3.49 (2 H, t, CH₂CH₂), 6.81 (2 H, m, ArH), 7.30 (1 H, t, ArH), 7.55 (3 H, br s, NH₃⁺), 7.90, (1 H, d, ArH), 8.51 (1 H, s, 4′-H) and 14.71 (1 H, s); 13C NMR [100 MHz, (CD₃)₂SO]δ_{C} 30.5, 37.3, 58.4, 117.5, 118.2, 119.9, 130.1, 133.3, 137.9, 145.9,161.3, 171.6 and 172.2.

### Example 6. 2-Hydroxybenzoyl-N-[(5-Chloro)Thiazol-2-yl]Amide, 3-Amino-1-Propanol Salt

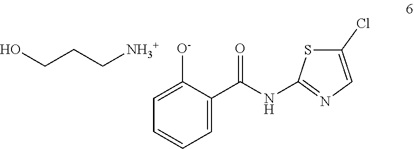 [SMILES: [NH3+]CCCO ; O=C(Nc1ncc(Cl)s1)c1ccccc1[O-]]

This was prepared similarly to 1 from RM4848 (2-hydroxybenzoyl-N-[(5-chloro)thiazol-2-yl]amide; 0.51 g, 2 mmol) and 3-amino-1-propanol (0.16 mL). The final methanol solution was concentrated and diluted with Et2O, leading to crystallization; the mixture was cooled to complete crystallization, then the solid was filtered, washed with Et₂O and dried to give the desired salt 6 (0.51 g, 77%). Found: C, 47.5; H, 5.0; N, 12.7; S, 9.6. C₁₃H₁₆ClN₃O₃S requires C, 47.3; H, 4.9; N, 12.7; S, 9.7%; 1H NMR [400 MHz, (CD₃)₂SO]δ_{H} 1.68 (2 H, m, CH₂CH₂CH2), 2.86 (2 H, t, CH₂CH₂), 3.49 (2 H, t, CH₂CH₂), 6.70 (2 H, m, ArH), 7.19 (1 H, dt, ArH), 7.22 (1 H, s, 4′-H) and 7.83 (1 H, dd, ArH); 13C NMR [100 MHz, (CD₃)₂SO]δ_{C} 30.5, 37.3, 58.4, 114.2, 116.8, 117.3, 120.4, 129.4, 132.0, 135.3, 162.3, 164.5 and 169.5.

### Example 7. 2-Hydroxybenzoyl-N-[(5-Nitro)Thiazol-2-yl]Amide, Diethanolamine Salt

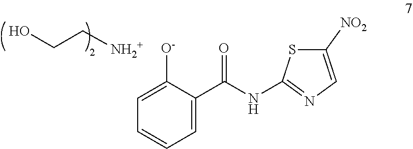

Tizoxanide (sc. 2-Hydroxybenzoyl-N-[(5-nitro)thiazol-2-yl]amide, 0.53 g, 2 mmol) was suspended in methanol (MeOH, 70 ml) containing diethanolamine (0.20 mL) and warmed until a virtually complete solution was obtained, then filtered. The clear filtrate was cooled, then concentrated, followed by cooling to 0° C. to complete crystallization. After filtration, washing with Et₂O containing a little MeOH and drying, the desired product was obtained in two crops, affording the title salt 7 (0.36 g, 49%) as a yellow crystalline solid. Found: C, 45.35; H, 4.9; N, 15.25; S, 8.7. C₁₄H₁₈N₄O₆S requires C, 45.4; H, 4.9; N, 15.25; S, 8.7%; 1H NMR [400 MHz, (CD₃)₂SO]δ_{H} 3.02 (4H, t, 2xCH₂CH₂), 3.66 (4H, t, 2xCH₂CH₂), 5.18 (2H, br s, OH), 6.81 (2H, m, ArH), 7.31 (1H, m, ArH), 7.91 (1H, m, ArH), 8.34 (2H, br s, NHs), 8.50 (1 H, s, 4′-H) and 14.70 (1H, br s, NH); 13C NMR [100 MHz, (CD₃)₂SO]δ_{C} 49.3, 56.7, 117.5, 118.2, 119.9, 130.1, 133.4, 137.9, 145.9, 161.3, 171.6 and 172.

### Example 8. 2-Hydroxybenzoyl-N-[(5-Chloro)Thiazol-2-yl]Amide, Diethanolamine Salt

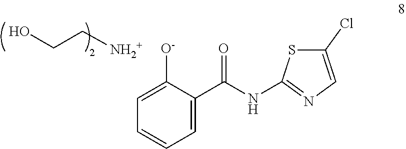

This was prepared similarly to 1 from RM4848 (2-hydroxybenzoyl-N-[(5-chloro)thiazol-2-yl]amide; 0.51 g, 2 mmol) and diethanolamine (0.24 mL) in MeOH (30 mL) with heating. A little insoluble material was removed by filtration, then the filtrate was concentrated followed by addition of Et2O. The mixture was cooled to complete crystallization, then the solid was filtered off, washed with Et₂O containing a little MeOH and dried to give the title salt 8 (0.545 g, 76%) as a near-white solid. Found: C, 46.9; H, 5.1; N, 11.8; S, 8.85. C₁₄H₁₈ClN₃O₄S requires C, 46.7; H, 5.0; N, 11.7; S, 8.9%; 1H NMR [400 MHz, (CD₃)₂SO]δ_{H} 3.00 (4 H, t, 2xCH₂CH₂), 3.65 (4 H, t, 2xCH₂CH₂), 6.70 (2 H, m, ArH), 7.20 (1 H, m, ArH), 7.23 (1 H, s, 4′-H) and 7.84 (1 H, dd, ArH); broad exchangeable peaks at δ5.17 (2 H) and δ8.2; 13C NMR [100 MHz, (CD₃)₂SO]δ_{C} 49.4, 56.8, 114.4, 116.9, 117.4, 120.3, 129.5, 132.2, 135.3, 162.2, 164.2 and 169.3.

### Example 9. 2-Hydroxybenzoyl-N-[(5-Nitro)Thiazol-2-yl]Amide, N-Methylpiperazine Salt

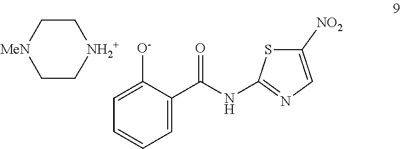 [SMILES: CN1CC[NH2+]CC1 ; O=C(Nc1ncc([N+](=O)[O-])s1)c1ccccc1[O-]]

Tizoxanide (0.50 g, 1.90 mmol, 1.0 eq) was suspended in methanol (10 mL) with N-methylpiperazine (0.90 mL). The mixture was heated with addition of further methanol (10 mL) to generate a clear solution. The mixture was then left to cool overnight. The resulting precipitate was filtered off to afford the final product as a yellow crystalline solid (0.13 g, 19% yield). Found: C, 49.0; H, 5.2; N, 19.0; S, 9.0. C₁₅H₁₉N₅O₄S requires C, 49.3; H, 5.2; N, 19.2; S, 8.8%; 1H NMR [400 MHz, (CD₃)₂SO]δ_{H} 2.23 (3H, s), 2.50-2.51 (4H, m), 3.06-3.09 (4H, m), 6.80-6.84 (2H, m), 7.32 (1H, t, J=7.7 Hz), 7.92 (1H, d, J=8.1 Hz), 8.52 (1H, s); 13C NMR [400 MHz, (CD₃)₂SO]δ_{C} 43.36, 45.79, 51.66, 117.48, 118.20, 119.85, 130.07, 133.38, 137.95, 145.91, 161.29, 171.50 and 172.16.

### Example 10. Bioavailability Pharmacokinetic Study of RM-5071 and RM-5072 Administered Orally in Rats

### Summary

A study was conducted to evaluate the bioavailability of tizoxanide (T) and tizoxanide glucuronide (TG) in plasma following oral administration of a single dose of 90 mg/kg RM-5071, RM-5072 and nitazoxanide by oral gavage to four male and four female Sprague-Dawley rats. Plasma samples were collected at 0.083, 0.167, 0.25, 0.5 1, 2, 6, 12 and 24-hours post-dose. Concentrations of T and TG were determined using mass spectrometry. No adverse clinical signs were observed for any of the rats in the three groups. R1\4-5071 and R1\4-5072 both dramatically improve the speed of availability of T and TG in plasma compared to NTZ. These compounds are rapidly absorbed achieving Cmax within 5 minutes after an oral dose. RM-5071 was associated with higher plasma concentrations of T and TG and less variability of absorption than either RM-5072 or NTZ.

### Introduction

Nitazoxanide (NTZ), a pro-drug for T and TG, is poorly absorbed following oral administration in animals and humans. Absorption is significantly affected by food, and there is significant intra- and inter-subject variability in T and TG concentrations. Two new salts of T, RM-5071 and RM-5072, were prepared to evaluate the possibility of improving bioavailability of T and TG following oral administration. This study was performed to evaluate the bioavailability of T and TG in plasma following administration of a single dose of 90 mg/kg RM-5071, RM-5072 and NTZ by oral gavage to Sprague-Dawley rats.

### MATERIALS AND METHODS

RM-5071 is 2-Hydroxybenzoyl-N-[(5-nitro)thiazol-2-yl]amide, ethanolamine salt. RM-5072 is 2-Hydroxybenzoyl-N-[(5-nitro)thiazol-2-yl]amide, morpholine salt. RM-5071 and RM-5072 were as disclosed above.

Animals and treatment. Three groups of four male and four female Sprague-Dawley rats were administered RM-5071, RM-5072 or NTZ as a single oral gavage dose as detailed in the table below:

TABLE 1

| | | Route of | | Dose | Dose | Dose |
|---|---|---|---|---|---|---|
| Group | No. of | Admin- | Com- | Level | Conc. | Volume |
| No. | Animals | istration | pound | (mg/kg) | (mg/mL) | (mL/kg) |
| 1 | 4M/4F | PO | RM-5071 | 90 | 6 | 15 |
| 2 | 4M/4F | PO | RM-5072 | 90 | 6 | 15 |
| 3 | 4M/4F | PO | NTZ | 90 | 6 | 15 |

Nine serial blood samples were obtained from each animal at 0.083, 0.167, 0.25, 0.5 1, 2, 6, 12 and 24-hours post-dose. Derived plasma samples were stored in sodium heparinized tubes at −70° C. or lower until they were shipped on dry ice via overnight courier for analysis of T and TG concentrations by mass spectrometry.

### Results

No adverse clinical signs were observed for any of the rats in the three groups.

Following administration of RM-5071, RM-5072 and NTZ, maximum concentrations (Cmax) of T (medians) were 4.7, 3.1 and 1.7 μg/mL, respectively. For RM-5071 and RM-5072, Cmax was reached at the first plasma sampling timepoint, 5 minutes after dosing. In the case of NTZ, the Cₘₐₓ of T in plasma (only 1.7 μg/mL) was achieved after 2 hours.

Because some animals glucurono-conjugate T faster than others, the extent, rate and variability of absorption of these three compounds using the sum of free and glucurono-conjugated T concentrations at each timepoint were evaluated. To arrive at the concentrations of glucurono-conjugated T, TG concentrations were multiplied by 61% (molecular weight of T=270 divided by molecular weight of TG=441).

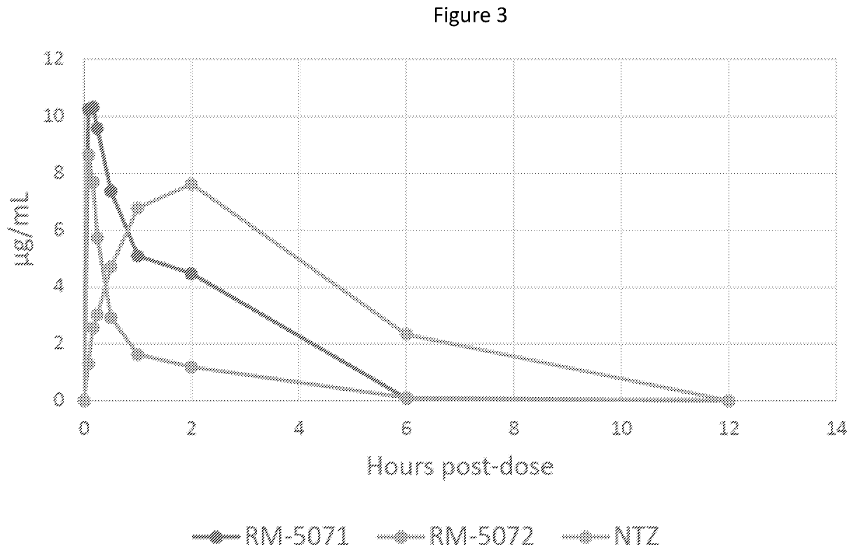

The sums of median free T plus glucuronidated T concentrations in plasma over the 12 hours post-dose are presented in FIG. 3.

Mean Cₘₐₓ and AUC₀₋₁₂ₕ values for the sum of free and glucurono-conjugated T are presented in Table 2 along with relative standard deviations (RSD). The mean Cₘₐₓ for RM-5071 was 33% and 47% higher than for RM-5072 and NTZ, respectively, with an RSD of 31% compared to 44% for both RM-5072 and NTZ.

TABLE 2
Mean Cₘₐₓ and AUC_{0-12 h} of the sum of
free and glucuronidated T in plasma

| | |
|---|---|
| Cₘₐₓ | AUC_{0-12 h} |

| | Mean | RSD¹ | Mean | RSD¹ |
|---|---|---|---|---|
| RM-5071 | 12.6 | 31% | 45.5 | 16% |
| RM-5072 | 9.5 | 44% | 25.0 | 36% |
| NTZ | 8.6 | 44% | 44.8 | 36% |

¹Relative standard deviation

The mean AUC₀₋₁₂ₕ for RM-5071 was almost double that of RM-5072, but it was roughly the same as that for NTZ. The comparison of AUC₀₋₁₂ₕ with NTZ is affected by the collection of only one plasma sample (the 6-hr sample) between 2 and 12 hours and the fact that NTZ is absorbed more slowly than the other compounds. The actual AUC₀₋₁₂ₕ value for NTZ would likely have been much lower had additional samples been collected—particularly between the 6 and 12-hour post-dose timepoints.

Notably, the RSD associated with the mean AUCO-12h for RM-5071 was only 16% compared to 36% for both RM-5072 and NTZ. This indicates that the inter-subject variability of absorption associated with NTZ is significantly improved by RM-5071.

### Conclusions

RM-5071 and RM-5072 both dramatically improve the speed of availability of T in plasma compared to NTZ. These compounds are rapidly absorbed achieving Cmax within 5 minutes after an oral dose. RM-5071 is associated with higher plasma concentrations of free and glucurono-conjugated T and less variability of absorption than either RM-5072 or NTZ. This study indicates that the rate, extent and variability of absorption is improved for RM-5071 compared to RM-5072 or NTZ.

### Example 11. Amine Salts of Thiazolides

A total of ten amine salts, five for each of tizoxanide and RM14848 were made.

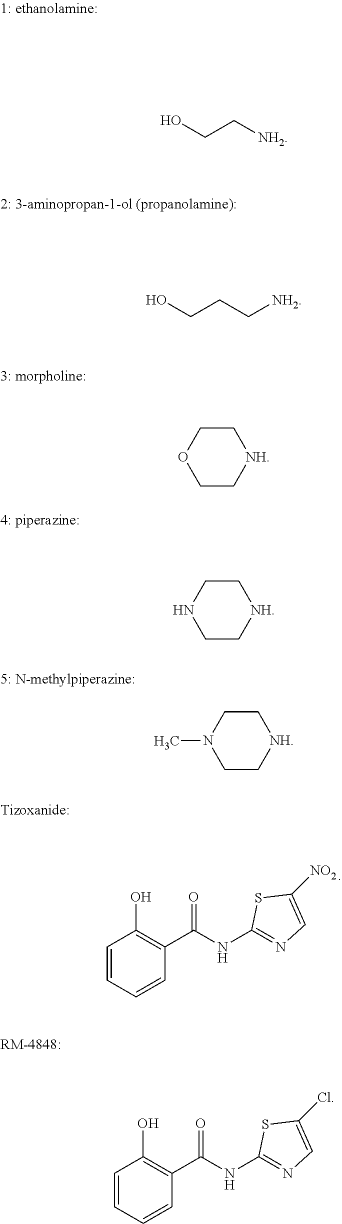

### General Procedure

The appropriate amine is heated with an equimolar amount of either tizoxanide or RM4848 in methanol until a clear solution is obtained. Any small amounts of undissolved solid are removed by filtration. On cooling, the desired salt may crystallize immediately; addition of an equal volume of a solvent, such as diethyl ether (for the tizoxanide salts) or concentration to a small volume, then addition of excess ether (for salts of RM4848), may be used to obtain solid products. In general the salts of RM4848 are more soluble under the above conditions. All the salts are more soluble in water than the parent thiazolides.

1: Both tizoxanide and RM4848 readily gave crystalline salts with ethanolamine (RM5071 and RM5072). These were obtained in good crystalline form and microanalytically pure. The term “microanalytically pure” may mean that deviation in an amount for each atom in a synthesized molecule from a respective theoretical value is within ±0.3% of the theoretical value.

2: Essentially the same as with ethanolamine. Good yields, crystalline form and chemical purity for both propanolamine salts.

3: The morpholine salt of tizoxanide was readily obtained in high purity. In the case of RM 4848, the first compound to separate as a solid was unchanged RM4848. Concentration of the mother liquors to a small volume afforded the desired salt in about 30% yield but high purity.

4: The piperazine salts of both tizoxanide and RM4848 were obtained by the standard method, but it was difficult to obtain them in satisfactory purity. Excess piperazine appears to co-crystallize with the salts. Although the present invention is not limited by its theory of operation, this may be due to the fact that piperazine is itself a solid.

5: Using N-methylpiperazine, a liquid, the tizoxanide salt was obtained in very good yield and purity. The corresponding salt of RM4848 has been obtained as a solid, but less pure.

### Synthesis of RM-5071 from Nitazoxanide

### Step 1: Preparation of Tizoxanide from Nitazoxanide

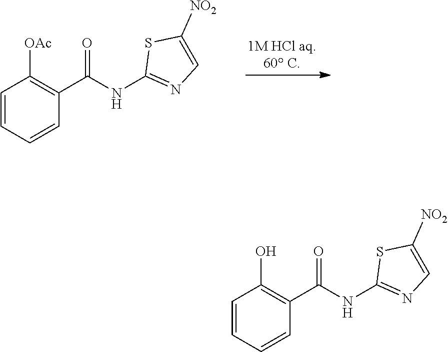 [SMILES: CC(=O)Oc1ccccc1C(=O)Nc1ncc([N+](=O)[O-])s1 ; O=C(Nc1ncc([N+](=O)[O-])s1)c1ccccc1O]

Nitazoxanide dissolved in a polar solvent, such as DMF, at for example 3Veq. The solution heated to an elevated temperature such as 50° C. An acid, such as HC1 1M, added at for example 1 Veq. The solution further heated to a second elevated temperature, such as 70° C., until a full conversion, which may take place over a period of time from about 36 hours to about 48 hours. The solution cooled down to room temperature and neutralized with a base, such as NaOH, at for example 1 M. The solution filtered and the produced cake washed with a solvent, such as water and/or alcohol, such as methanol. The reaction allows recovering 90-100% of tizoxanide with a good purity. The reaction is suitable for upscaling.

### Step 2: Preparation of RM5071 from Tizoxanide

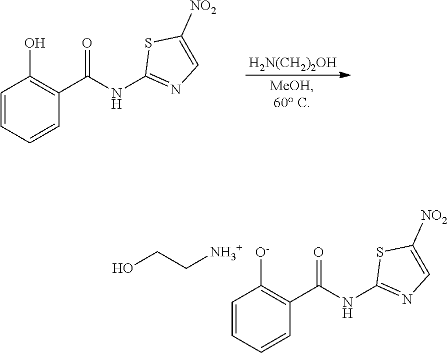

Tizoxanide dispersed at room temperature in a solvent, which may be an alcohol, such as methanol, at for example 5Veq. Ethanolamine slowly added at for example, 1.1 eq. Exotherm. The mixture stirred for, for example, about 2 hours. The mixture then filtered the produced cake washed with a solvent, such as methanol and ethylacetate, which may be at about 1:1 volume ratio. The cake dried solid under vacuum, such as below about 100 mbar, at an elevated temperature, such as about 60° C. The dried solid may be milled and/or crushed. The reaction allows recovering 80-90% of RM-5071 with a good purity. The reaction is suitable for upscaling.

### Summary

RM-5071 may be synthesized from nitazoxanide is a two-step synthesis. Conditions for making RM-5071 may be compatible with upscaling in production facilities because they use limited dilution, mild conditions and product recovery by centrifugation. The product is usually obtained with a good purity. 2 purification possibilities in case of bad purity. A yield may be 80 - 85 g RM5071 from 100 g Nitazoxanide. Exemplary upscaling conditions could be as follows:

| Reactor Scale | Nitazoxanide | RM-5071 |
|---|---|---|
| 15 L | 1 kg | 0.8 kg |
| 500 L | 50 kg | 40 kg |
| 6000 L | 600 kg | 500 kg |

### Analytical Information for RM-5071

| | RM5071 | Tizoxanide |
|---|---|---|
| Melting point | 140-160° C. | 240-250° C. |
| FTIR (as solid) | Specific signals from | v (O—H) = 3250 cm⁻¹ |
| | Tizoxanide not detected | v (C═O amide) = 1650 cm⁻¹ |
| UV (λₘₐₓ in solution) | 410-440 nm | 430 nm |
| | 290 nm (small | 360 nm |
| Elemental analysis | 44.1% C (exp.) | 44.9% C (calc.) |
| | 4.3% H | 3.4% H |
| | 17.2% N | 15.7% N |
| | 9.8% S | 12, -% S |

### Example 12

### Abstract

The thiazolides, typified by nitazoxanide (NTZ), viz. 2-[(acetyloxy)-N-(5-nitro-2-thiazolyl)] benzamide, are an important class of polypharmacology agents, which may have a wide range of antiinfective activities. The prototype NTZ, originally marketed as an antiparasitic agent especially against Cryptosporidium spp., was subsequently shown to be effective against a number of viruses. Nevertheless, the pharmacokinetic parameters of NTZ are not ideal in cases where efficient systemic circulation is required because of its poor solubility and absorption. This study reports the preparation and evaluation of a series of amine salts of tizoxanide, the active deacetyl metabolite of NTZ, and the corresponding 5-Cl thiazolide RM4848. The thiazolide salts indeed demonstrated improved aqueous solubility and absorption as shown by in vivo measurements and have lately been scaled up for clinical trials.

### Introduction

Nitazoxanide [NTZ; 2-[(acetyloxy)-N-(5-nitro-2-thiazolyl)] benzamide] 1a was first reported in 1976 by Rossignol and Cavier;¹ it was modelled on the known antiinfective agent niclosamide 2,² replacing the anilide by a nitrothiazolyl amide and showed promising antiparasitic activity in vitro and in vivo. Originally NTZ la was developed for the treatment of protozoal and helminth parasitic infections,^{3, 5} but later its most important application became the treatment of Cryptosporidium spp. infections:^{6, 7} to this day, it is the only FDA-approved treatment for Cryptosporidium parvum. It has been established from studies of its antiparasitic activity that one important mode of action of NTZ 1a is inhibition of the folding chaperone protein disulfide isomerase.⁸ NTZ 1a also has valuable antibacterial activity against both aerobic and anaerobic species, operating by inhibition of pyruvate oxidoreductases in the case of anaerobes.^{9, 10}

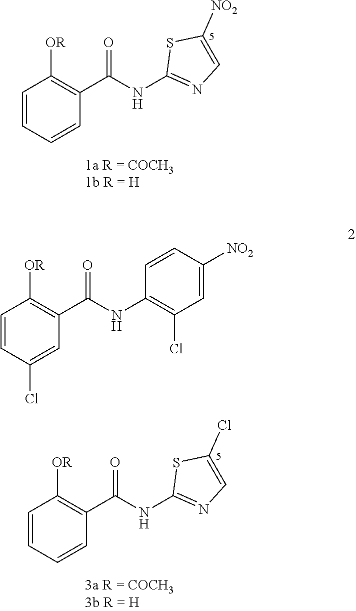

NTZ 1a was discovered to have an antiviral activity, during the course of treating cryptosporidiosis in patients with AIDS.¹¹ The first clinical trial with NTZ 1a as an antiviral agent was against rotavirus-induced diahorrea,¹² including young children as patients. NTZ 1a proved to have an antiviral activity against a number of viruses¹³⁻¹⁶

The nitro group may be not essential for activity: the 5′-Cl analogue 3a may have an almost parallel spectrum of activity, at low micromolar values,18 and the 4′-ethanesulfonyl analogue 4 shows excellent in vitro activity against an H1N1 strain of influenza A virus, ICso=0.14 μM.¹⁹⁻²¹

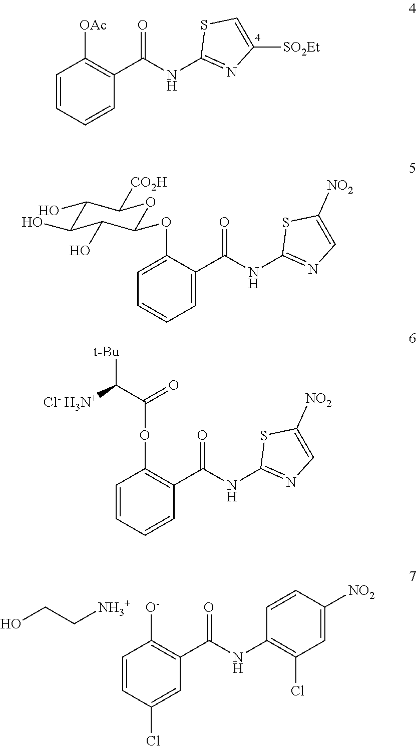

NTZ 1a is usually administered orally but is only partially absorbed from the gastro-intestinal tract.' It is effectively a prodrug for the deacetyl derivative tizoxanide 1b, which is formed immediately on absorption and subsequently excreted from the body largely as the O-glucuronide 5:²³ 1a has a plasma half-life of 1.3 h. Such a biodisposition may be acceptable for intestinal infections, but to achieve adequate systemic circulation of 1a/1b for viral infections, such as influenza A may be challenging. Prodrug amino-acid esters such as 6 which may improve the absolute oral bioavailability of 1a, to about 20% in the case of 6.²⁴ An alternative approach to improve the pharmacokinetic parameters of 1a/1b is to administer the active agent as an amine salt. This study reports the synthesis of a set of amine salts of thiazolides 1b and RM4848 3b, their characterisation and selected pharmacokinetic data. In general, not all amines we selected gave satisfactory results and the behaviour of 1b and 3b was different in some cases.

### Discussion

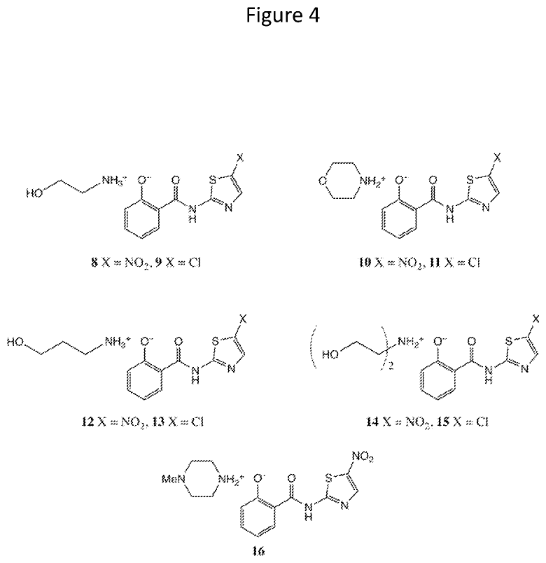

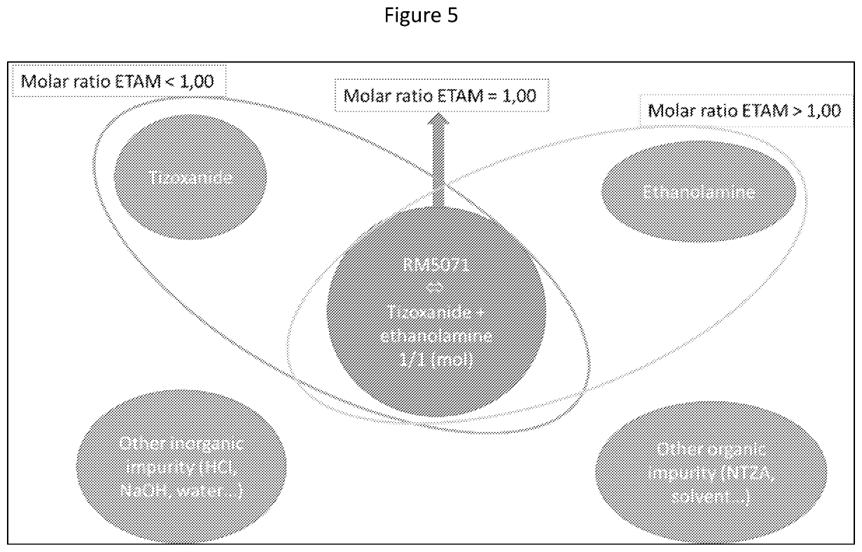

When tizoxanide 1b was heated in methanol with a slight excess of ethanolamine for about 0.25 h, an almost clear solution was obtained. Filtration followed by concentration led to crystallisation of the desired salt 8; after dilution with diethyl ether, cooling and filtration, 8 was obtained in good yield and excellent microanalytical purity. The 1H NMR showed a characteristic upheld shit of the aryl protons, consistent with the anionic nature of the thiazolide. Similarly, RM4848 3b afforded salt 9. FIG. 4 summarises a total of nine salts made similarly, employing hydroxyamines, morpholine and N-Me piperazine. Some significant differences were noted with specific thiazolide/amine combinations. Thus the morpholine salt 10 was obtained in the normal manner from 1b, but when RM4848 3b was used the first solid to precipitate was unreacted 3b. Concentration of the filtrate led to the desired salt 11, inevitably in rather low yield but still microanalytically pure. Salts 12 to 15 were similarly obtained using 1-aminopropanol (12, 13) and diethanolamine (14, 15). Diamines proved more difficult to handle, and from piperazine pure salts could not be easily obtained; piperazine is a solid and difficult to remove by recrystallization. However, from N-Me piperazine, a liquid, and 1b the salt 16 could be obtained although in low yield; the site of protonation in this case was not determined. The amino-acid L-lysine did not give an isolable salt from either 1b or 3b.

### Upscaling

Synthesis of ethanolamine salt 8 was successfully scaled up to an industrial process. It consists in a two steps synthesis (80% yield) starting from FDA approved drug Nitazoxanide 1a. A pre-technical batch was prepared yielding into 40 kg of pure material. Using production plant equipment it was demonstrated that the process is reliable for large scale manufacturing.

### Pharmacokinetics

Pharmacokinetics data is presented in Example 10.

### Experimental

### General experimental methods

Salts were prepared as outlined in Examples 1-9.

1H and 13C spectra were obtained on a Bruker 400MHz instrument (100 MHz for 13C spectra) equipped with a multinuclear 5-mm BBFO probe. 1H spectra (at 400.13 MHz) and 13C(1H) spectra (at 100.61 Mhz) were acquired at ambient temperature using standard parameters set; solvent resonances were used for referencing purpose.

Low- and high-resolution mass spectra were obtained by direct injection of sample solutions into a Micromass LCT mass spectrometer operated in the electrospray mode, by +ve or −ve ion as indicated (Micromass LCT Waters Micromass UK Ltd, Manchester, UK).

### REFERENCES

1. J.-F. Rossignol and R. Cavier, Chem. Abstr., 1975, 83, 28216n.
2. G. J. Frayha, et al, Gen. Pharmacol., 1997, 28, 273-299.
3. R. Cavier and J. -F. Rossignol, Rev. Med. Vet., 1982, 133, 779-783.
4. L. M. Fox and L. D. Saravolatz, Clin. Infect. Dis., 2005, 40, 1173-1180.
5. J.-F. Rossignol and H. Maisonneuve, Am. J. Trop. Med. Hyg., 1984, 33, 511-512.
6. O. Doumbo, et al., Am. J. Trop. Med. Hyg., 1997, 56, 637-639.
7. J.-F. Rossignol, A. Ayoub and M. S. Ayers, J. Infect. Dis., 2001, 184 103-106.
8. J. Muller, et al., Exp. Parasitol., 2008, 118, 80-88.
9. L. Dubreuil, et al., Antimicrob. Agents Chemother., 1996, 40, 2266-2270.
10. P. S. Hoffman, et al., Antimicrob. Agents Chemother., 2007, 51, 868-876.
11. J.-F. Rossignol, Aliment. Pharmacol. Ther., 2006, 24, 887-894.
12. J.-F. Rossignol, et al., Lancet, 2006, 368, 124-129.
13. J.-F. Rossignol and E. B. Keefe, Future Microbiol., 2008, 3, 539-545.
14. J.-F. Rossignol, et al, Aliment. Pharmacol. Ther., 2008, 28, 574-580.
15. J. Haffizulla, et al., Lancet Infect. Dis., 2014, 14, 609-618.
16. J.-F. Rossignol, Antiviral Res., 2014, 110, 94-103.
17. NIH Website clinicaltrials.gov NCT04341493.
18. B. E. Korba, et al., Antiviral Res., 2008, 77, 56-63.
19. A. V. Stachulski, et al J. Med. Chem., 2011, 54, 4119-4132.
20. A. V. Stachulski, et al., J. Med. Chem., 2011, 54, 8670-8680.
21. A. V. Stachulski, et al., Future Med. Chem., 2018, 10, 851-862.
22. J. Broekhuysen, et al. Int. J. Clin. Pharmacol. Ther., 2000, 38, 387-394.
23. J.-F. Rossignol and A. V. Stachulski, J. Chem. Res. (S), 1999, 44-45.
24. A. V. Stachulski, et al., Eur. J. Med. Chem., 2017, 126, 154-159.
25. G. Hecht and C. Gloxhuber, Z. Tropenmed. Parasit., 1962, 13, 1-8.
26. P. Andrews, et al., Pharmac. Ther., 1983, 19, 245-295.
27. R. Krieger and W. Krieger, Handbook of pesticide toxicology, 2001, 1225-1247.
28. H. Tao, et al, Nat. Med. 2014, 20,1263-1269.
29. D. Lu, et al., Xenobiotica, 2016, 46, 1-13.
30. M. A. Gemmell, et al., Res. In Vet. Sci., 1977, 22, 389-391.

### ADDITIONAL REFERENCES

Rossignol, J. F. Antiviral Res 2014; 110:94-103.
Wang, M., et al. Cell Research (2020) 0:1-3; https://doi.org/10.1038/s41422-020- 0282-0
Rossignol, J. F. & Van Baalen, C. Poster presentation at the 2nd International Meeting on Respiratory Pathogens March 7-9, 2018, Abstract ARP057 page 19
Braakman, I., et al. Nature 1992; 356:260-62
Braakman, I., et al. J. Cell Biol. 1991; 114:401-11
Doms. R. W., et al. J. Cell Biol 1987:105:1957-69
Chang, C. W., et al., J. Biomedical Sci 2009; 16:80
Mirazimi A, Svensson L VP7 J Virol, 2000; 74: 8048-52
Rossignol, J. F., et al., Journal of Biological Chemistry. 2009; 284:29798-29808.
Piacentini S, et al., Research Report, 2018;8: 10425
Cao J, Forest C, Zhang X Antiviral Res, 2015; 114: 1-10
Lee, J. H., et al., Int J Obes 2017; 41: 645-51
Sag, D., et al., J. Immunol 2008; 181:8633-41
Wang, W., et al., J. Biol Chem 2003: 278:27016-23
J. Ditzel and M. Schwartz, Acta Med. Scand., 1967, 182, 663-664.
R. A. Mook, et al., Bioorg. Med. Chem., 2015, 23, 5829-5838.
A. Jurgelt, et al., PLOS Pathogens, 2012, 8, e1002976.

### Example 13

RM5071 is a prodrug of Tizoxanide (TIZ), the active metabolite of Nitazoxanide (NTZA), an antiprotozoal drug called Alinia approved by the FDA. RM5071 is an organic salt composed of two moieties: Tizoxanide and ethanolamine (ETAM). There is a need for compounds with similar activity as NTZA, but with a greater oral bio disposition and metabolism which will liberate in the blood stream Tizoxanide. A pharmacokinetics (PK) study performed in Sprague-Dawley rats showed that RM5071 is more bioavailable in terms of the maximum concentration than NTZA. There may be a need to develop an up scalable synthetic process for RM5071.

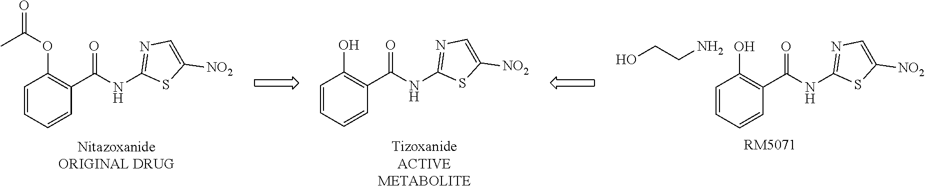

| Term | Definition |
|---|---|
| DMF | Dimethylformamide |
| DS | Drug Substance |
| ESI | Electro Spray Ionization |
| ETAM | 2-ethanolamine |
| FDA | Food and Drug Administration |
| FTIR | Fourier-Transform Infra Red spectroscopy |
| HCl | Hydrochloric acid |
| HPLC | High Performance Liquid Chromatography |
| LCMS | Liquid Chromatography with Mass Spectrometry detection (HPLC-MS) |
| LOD | Loss On Drying |
| MS | Mass spectrometry |
| NaOH | Sodium hydroxide |
| NMR | Nuclear Magnetic Resonance |
| NTZA | Nitazoxanide |
| PK | Pharmacokinetic |
| QC | Quality Control |
| qNMR | Quantitative NMR |
| RT | Room Temperature (+15/+25° C.) |
| THF | Tetrahydrofuran |
| TIZ | Tizoxanide |
| UV | Ultra Violet |
| Veq | Volume equivalents (1Veq = 1 L solvent per kg of starting material |

### 1. Discussion

### 1.1. Initial Approach

RM5071 was originally prepared by the following protocol (see also Appendix 1): 2 mmol Tizoxanide were suspended in 20 mL methanol containing 0.15 mL ethanolamine. The suspension was warmed to +50° C. for a few minutes, filtered and the filtrate was concentrated to 5 mL. Crystallization readily began, diethyl ether (5 mL) was added and mixture was cooled to 0° C. prior filtration. The cake was washed with diethyl ether containing a little methanol. Drying afford RM5071 as a yellow crystalline solid (0.49 g).

Nuclear Magnetic Resonance NMR (NMR 1H and 13C), elemental composition, and Electrospray Ionization Mass Spectrometry (MS-ESI negative) confirmed the expected structure. Melting point was measured between 158° C. and 160° C. (decomposition).

This batch of RM5071 was used for the first toxicology/PK study and was later used as a reference for the process development.

### 1.2. Development of the Scalable Process

The development of the drug substance (DS) synthesis was performed in 3 steps. Starting from the initial synthesis, several lab scale tests were performed. They lead to a scalable process, which was then tested at a larger scale in the pilot lab. Since no issues were reported during this pilot lab test, an engineering batch was prepared. For that larger batch the idea was to confirm if the process was effectively doable using current production equipment.

Criteria for scaling up RM-5071 synthesis may include one or more of the following:
Yield
Time
Safety
Purity, such as a ratio ETAM/TIZ.

Constraints may include working temperature (between −5° C. and +80° C.)

### 1.2.1. Theoretical Considerations and Synthesis Strategy

RM5071 is the ethanolamine salt of Tizoxanide (TIZ). The alcoholic site from TIZ (phenol) is slightly acidic. This characteristic is used to form an organic salt by combining TIZ with an alkaline molecule: 2-ethanolamine (ETAM). From a reactivity point of view, ethanolamine is not a strong base (compare to other organic bases), however, it may be strong enough to have an interaction with the slightly acidic phenol function of TIZ. Hence, the salt may be formed by mixing together both molecules.

Due to this strong interaction between ethanolamine and Tizoxanide, RM5071 chemical properties differ from Tizoxanide's chemical properties. Accordingly, a change in melting point (degradation) as well as changes in FTIR spectra have been observed. Such differences may be possibly linked to the inter-molecular arrangement.

The synthesis plan for RM5071 may be thought as a two steps synthesis starting from Nitazoxanide (NTZA), with the second step being the salt formation which produces RM5071.

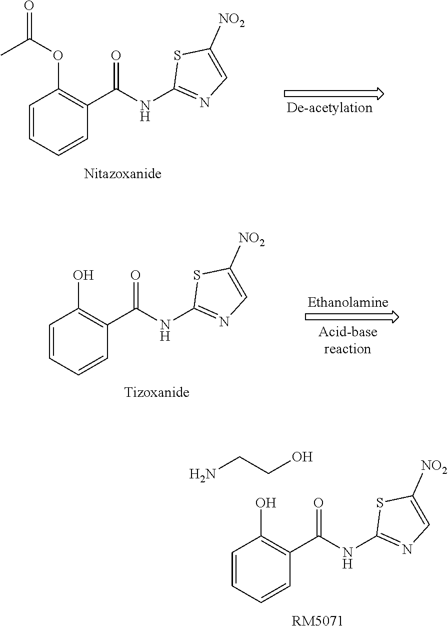

### 1.2.2 Small Scale Tests

### 1.2.2.1 Step 1

Tizoxanide preparation from NTZA was already previously studied. One process protocol may be the following:

Disperse NTZA in 10 Veq HCl 37% at RT, forming a very thick yellow suspension. Heat the mixture to +50° C. during 24 hours (until full conversion). Slurry becomes less thick and better stirrable. After cooling to RT, it is diluted twice with water (10 Veq) and filtered. The cake is washed with plenty of water, then with methanol. Yellow cake is dried under vacuum to afford a quantitative yield of pure Tizoxanide.

This process may have two issues, making it not easily up scalable. Firstly, this process uses concentrated HCl, which is better to avoid on large scale for safety reasons. Secondly, this process may need a large reactor, since after dilution with water, a total of 20 Veq are needed. This may mean that with a 6000 L reactor, a maximum of 300 kg of NTZA could be used in one batch.

During previous projects, reaction in organic media was tried, i.e. dissolving NTZA in THF and letting it react with aqueous ammonia. This reaction was finished in a few minutes and did not need heating. However, the work up process was not easily up scalable: concentration to dryness (evaporation of ammonia), reslurry in water, and acidification with HCl prior filtration. Several process variations were tested; however, this process was abandoned.

Efforts were focused on the hydrolysis with HCl. It quickly appears that in pure aqueous media no improvement is possible, i.e. working with lower HCl concentration leads to long reaction time even with an increase in temperature and working with lower dilution leads to a not stirrable mixture.

In parallel the reaction was also tested in DMF, as NTZA is soluble in DMF and slowly degrades into Tizoxanide. However the reaction kinetic was really slow, even when the temperature was increased and water was added as a catalyst in excess. Conversely, addition of a significant quantity of aqueous HCl together with heating leads to a reasonable speed of conversion. A brief description of the protocol is provided below:

Dispersed NTZA in DMF (3 Veq) at RT, add aqueous HCl (2 Veq 1M), heat at +70° C. until full conversion. Filtration and washings (water then methanol) afford quantitative yield after drying.

NTZA is almost soluble in DMF at the working concentration and room temperature; however, once the aqueous media was added, the mixture became very difficult to stir until the internal temperature reached about +50° C. The stirrability of the mixture increased with conversion of NTZA into Tizoxanide. Finally, the process was changed so the starting solution is heated to +50° C. before addition of the acid. No stirring issues were observed during the addition of HCl during the reaction.

In order to avoid handling of the acidic mixture and to prevent corrosion of the centrifuge in the plant, a neutralization step, by filtration in the lab but centrifugation in plant, was added before recovery of the solid Tizoxanide. Adding the same amount of NaOH 1 M (aqueous) to the mixture after the end of the reaction allow the neutralization of the free HCl in the reaction mixture. However, the reaction mixture stays acidic since the reaction generated 1 equivalent of acetic acid.

The resulting slurry was filtered without issues; the filtration went easy on a glass sintered funnel. The cake was washed with water (3×2 Veq) and methanol (3×2 Veq). Combining the neutralization step and aqueous washings provide that no HCl remains in the cake. Avoiding residual HCl in the product may be important because it could react with ethanolamine during step 2. Washing the cake with methanol allows for the removal of water. However, NTZA and Tizoxanide are fairly insoluble in water as well as in methanol. This way no Tizoxanide is lost during the washing of the cake; nevertheless, the remaining NTZA is not removed either.

In conclusion, an up scalable process for the preparation of Tizoxanide from NTZA was obtained.

Dispersed NTZA in 3 Veq DMF at RT. Heat to +50° C., add 2 Veq of HCl 1 M (aq.). Then heat to +70° C. until full conversion. Cool to RT, add 2 Veq NaOH 1M (aq.), filter and wash cake with 3×2Veq water and 3×2Veq methanol. Cake is dried under vacuum to give Tizoxanide as an off white powder.

Yield is quantitative and purity is good (100% UV area according HPLC). Process seems robust except for reaction time, which varied between 16 and 40 hours. Temperature of the reaction mixture may be important for the speed of conversion. Below +70° C. (internal temperature) the reaction time is longer. A special attention is taken to that point during pilot lab scale up. Other attention points are stirrability and the filtering ability of the reaction mixture, as they are known parameters on which scale up may have an impact.

### 1.2.2.2 Step 2

The earlier process was reproduced as a starting point for the process optimization. The earlier process gives the product with good purity but with low yield (36%). Several steps from that process are not suitable for large scale production: hot filtration, distillation, use of diethyl ether as well as large dilution (working concentration 0.1 M or 38 Veq). The heating and filtration steps may not be needed, unless some free Tizoxanide stays in suspension, but that may be unlikely given the high dilution. Hence, the new protocol was designed to avoid the heating and filtration steps, as well as the concentration and co-solvent crystallization step.

Tizoxanide was dispersed in an organic solvent, ethanolamine was added and the resulting slurry was filtered and washed with a solvent. Several tests showed that methanol is a good solvent for that process and can be used at a working concentration of 0.75 M (or 5 Veq). However, the final product is slightly soluble in methanol, so it was found that washing the cake with a mixture of methanol and ethyl acetate (1/1 v/v) gives good results.

RM5071 is not soluble in ethyl acetate, but it tends to form a sticky material. This was also observed during a test reaction in pure ethyl acetate. In addition, it was observed that RM5071 is almost soluble in methanol at a concentration of 5 mg/mL.

During the development of step 2 of the synthesis, time was also dedicated to the analysis of the final material. As a final material, an organic salt is composed of the 2 starting materials from that step, analysing it may be not as easy as in a classical organic reaction. This topic is discussed below later.

Even though no need for purification was observed, still it was studied. Two methods were tested: slurry and dissolution/crystallization.

Slurry was tested in water and in methanol, because both are expected to remove possible traces of ethanolamine. HCl salt as well as free ethanolamine. Residual Tizoxanide could not be removed this way. Both solvents showed an increase on purity (ETAM/TIZ ratio) and good yield:

TABLE 3
qNMR molar ETAM/TIZ ratio (n ETAM/n TIZ)
on 3 batches, before and after reslurry

| | Purified batch with methanol | Purified batch with water |
|---|---|---|
| Starting batch | method (purification yield) | method (purification yield) |
| 1.16 | 0.97 (92%) | 0.92 (84%) |

Dissolution/crystallization was not tested again in methanol, but in DMF:
RM5071 was dissolved in a minimum of DMF (2.5 Veq) at RT. Eventual solids can be filtered, however no solid is observed. Adding 25 Veq of ethyl acetate allows RM5071 to precipitate at RT. After filtration, product is washed with methanol/ethyl acetate (1/1).

Purification yield was about 70%. Excess of ethanolamine is removed but otherwise no gain is observed, compared to other purification methods or even no purification.

The reaction was also tried in DMF, with recovery of the product by co-crystallization with ethyl acetate. Analysis of the final material compared to other batches but high dilution is needed for the crystallization so this method may not show any advantage.

In conclusion, the methanol suspension reaction followed by simple filtration was kept for step 2:
Tizoxanide is dispersed in methanol (5 Veq) at RT. Ethanolamine (1.1 eq) is added resulting in a limited exotherm. Mixture is stirred 2 hours prior filtration and washing with a mixture methanol/ethyl acetate (1/1 V/V).

Yield is around 80%. Analysis shows good purity, with an ETAM/TIZ ratio between 0.90 and 1.00.

If final material contains an excess of ethanolamine or another salt, it may be purified by reslurry in methanol and/or water.

As free Tizoxanide was detected in the final material, a larger excess of ethanolamine was also tried. No differences were observed in the final material. A non-limiting hypothesis may be that traces of ethanolamine may be dissociated from Tizoxanide during the washings steps (filtration), resulting in this small excess of Tizoxanide.

### 1.2.2.3 Conclusion

An up scalable two steps process from NTZA to RM5071 has been developed.

### Step 1: Preparation of Tizoxanide from NTZA

Nitazoxanide is dispersed in 3Veq DMF, then mixture is heated to +50° C. HCl 1M (aqueous solution, 2 Veq) is added slowly. Mixture turns from yellow solution to white suspension. It is heated to +70° C. until conversion is completed (generally 36-48 hours).

Mixture is cooled to RT and NaOH 1M (aqueous, 2 Veq) is slowly added in order to neutralize HCl. Temperature needs to be controlled and cooling will be needed on large scale.

The suspension is filtered and the solid is washed 3× with 2Veq water, then 3× with 2Veq methanol. The solid is dried under vacuum to afford pure Tizoxanide.

### Step 2: Preparation of RM5071 from Tizoxanide

Tizoxanide is dispersed in 5 Veq methanol at RT. Ethanolamine (1.1 eq) is slowly added, keeping internal temperature below +30° C. Cooling may be needed on a larger scale. The suspension turned from off white to yellow. After 2 hours stirring at RT, the mixture is filtered and solid is washed 3 times with 2 Veq a mixture of methanol and ethyl acetate (1/1 V/V). Yellow powder is dried under vacuum (oven).

Solid is crushed to afford a nice yellow powder.

Upscaling criteria was met:
Yield: 80-90% over 2 steps.
Time:
Step 1: reaction time may be between 20 and 46 hours heating. This parameter may be further improved.
Step 2: reaction time may be set at 2 hours but chemically speaking, formation of RM5071 is instantaneous
Safety
   No use of concentrated acids/base
   No handling of strong acidic or alkaline mixture
   Some exotherm to control
   No use of volatile solvent
Purity
   HPLC-UV, HPLC-MS showed good purity
   qNMR showed an acceptable ratio ETAM/TIZ
   Particle size distribution looked good
Working temperature (between −5° C. and +80° C.)
   Cooling to maintain temperature around RT may be needed during some exothermic additions
   Heating to +70° C. (internal) may be achievable.

According to the selected process, the following quantities could be prepared:

TABLE 4
Summary of exemplary quantities for up scalable process

| | | | | | In production | | In production | |
|---|---|---|---|---|---|---|---|---|
| Scale | In pilot lab | | In pilot plant | | plant | | plant (max) | |
| Volume reactor | 16 | L | 500 | L | 5000 | L | 6000 | L |
| Scale (NTZA) | 1.5 | kg | 50 | kg | 530 | kg* | 700 | kg |
| Volume needed step 1 | 12 | L | 400 | L | 4240 | L | 5600 | L |
| m expected TIZ | 1.25 | kg | 43 | kg | 450 | kg | 600 | kg |
| m ETAM needed | 0.33 | kg | 11 | kg | 114 | kg | 152 | kg |
| Volume needed step 2 | 8.1 | L | 270 | L | 2815 | L | 3750 | kg |
| m expected RM5071 | 1.35 | kg | 44 | kg | 470 | kg | 625 | kg |

*Average mass of one NTZA batch 1. 2. 3 Pilot lab test

As a second part to the development of the process, the selected process was tested on a pilot lab scale. Two batches were prepared: one using classical glassware and one a pilot glass reactor.

Below are the main conclusions regarding the synthetic process optimization:
The addition of reagents (HCl, NaOH and ETAM) did not give problematic temperature increases. However, sufficient cooling may be needed when adding NaOH and ETAM (internal temperature cooling is set at +10° C.).
The reaction time during step 1 may be around 45 hours. A minimum temperature inside the reactor of +70° C. may be needed to achieve this reaction time. Temperatures below this level will lead to longer reaction times.
The filtration cloth of 50 p.m can be used for filtration in the centrifuges.
No difficulties regarding stirring (158 rpm, Buchi reactor) were noticed. The crucial part may be during step 1 when almost full conversion is reached.
The product was easily unloaded after both steps, not much residual stayed behind.

The reactor is easily cleaned.
The intermediate (Tizoxanide) is not dried before loading it back into the reactor to perform step 2. The Loss on Drying (LOD) has been measured. Depending on the LOD result, the quantity of methanol can be adjusted before loading it in step 2.

On 1.5 kg starting material scale yield was 88% and purity was conform to other development batches. Since there was no need to modify critical parameters, it was decided to directly test the process at a larger scale (50 kg starting material).

### 1.2.4. Engineering Batch

The main purpose of the engineering batch was to test the suitability of the process to manufacture RM5071. Parameters to check were the ability to heat the reaction mixture in step 1 at an efficient temperature (+75° C. internal), and the ability to recover Tizoxanide (step 1) and RM5071 (step 2) during centrifugation.

Conclusions regarding the synthesis development are as follows
Yield: 78% over 2 steps.
Time:
   Step 1: 20 hours heating; 32 hours total manufacturing time.
   Step 2: 2 hours reaction time; 7 hours total manufacturing time.
Safety
   Exothermic reactions were easily controlled
Purity
   No deviation observed compared to previous batches
Working temperature (between -5° C. and +80° C.)
   Cooling power was sufficient
   Heating at +75° C. was achieved. Full heating power was used.

In conclusion, 41 kg of RM5071 drug substance were prepared as an engineering batch following the synthesis process developed at pilot lab scale.

### 1.3. Analytical Consideration

The analysis of the final RM5071 was challenging. RM5071 is an organic salt composed of the Tizoxanide and Ethanolamine. Hence, classical analytical technics cannot be directly used to qualify the final product. There is a need for a method able to distinguish free reagents (ETAM and TIZ) from the final product (RM5071). During synthesis development determination of the ratio ETAM/TIZ was chosen for that purpose:

This molar value should be 1.00 accordingly the chemical structure of RM5071. If free Tizoxanide or ethanolamine is present in the final product the observed ratio will change allowing the quantification of the residual starting material, as drawn in 5. Other organic impurities are easier to determine (HPLC, NMR). Inorganic impurities are not expected in final product.

In addition to the final product, analysis of the intermediate (Tizoxanide) was also important, both to monitor the conversion during step 1 and to start final step with a pure material. An LC-MS method was used, allowing good trust in the results as well as quick results and easy sample preparation.

An important parameter for thiazolide, particle size distribution was also checked by laser diffraction during synthesis development to ensure process changes do not impact this distribution.

### 2. Conclusion

In conclusion, RM5071 drug substance can be prepared from lab scale to production scale using the following synthesis protocol:

### Preparation of Tizoxanide from NTZA (Step 1)

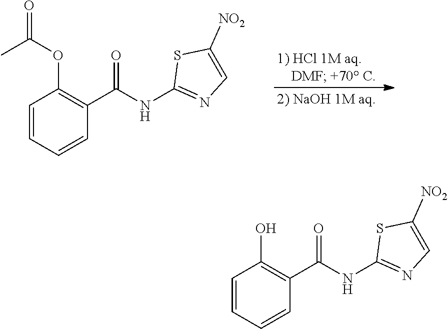 [SMILES: CC(=O)Oc1ccccc1C(=O)Nc1ncc([N+](=O)[O-])s1 ; O=C(Nc1ncc([N+](=O)[O-])s1)c1ccccc1O]

Nitazoxanide is dispersed in 3Veq DMF, then mixture is heated to +50° C. HCl 1M (aqueous solution, 2 Veq) is added slowly. Mixture turns from yellow solution to white suspension. It is heated to +75° C. until conversion is completed.

Mixture is cooled to +10/+15° C. and NaOH 1M (aqueous, 2 Veq) is slowly added in order to neutralize HCl, keeping internal temperature below +25° C.

The suspension is centrifuged (or filtered at small scale) on 50 μm filter cloth and the solid is washed 3 times with 2Veq water, then 3 times with 2Veq methanol. The solid is directly used in the next step without further drying.

### Preparation of RM5071 from Tizoxanide (Step 2)

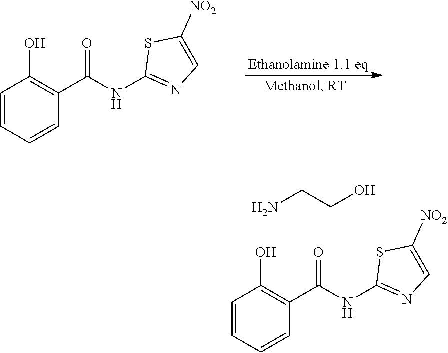

Tizoxanide is dispersed in 5 Veq methanol at RT. Mixture is cooled to +10/+15° C. Ethanolamine (1.1 eq) is slowly added, keeping internal temperature below +25° C. The suspension turned from off white to yellow. After 2 hours stirring at RT, the mixture is centrifuge (filtered at lab scale) on 50 μm filtercloth and solid is washed 3 times with 2 Veq a mixture of methanol and ethyl acetate (1/1 V/V). Yellow powder is dried under vacuum (+50° C., <100 mbar). Solid may be crushed if needed to afford RM5071 as a nice yellow powder.

### Appendix 1: Original protocol RM5071

### 2-Hydroxybenzoyl-N-[(5-Nitro)Thiazol-2-yl]Amide, Ethanolamine Salt

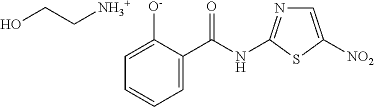

Tizoxanide (sc. 2-Hydroxybenzoyl-N-[(5-nitro)thiazol-2-yl]amide, 0.53 g, 2 mmol) was suspended in methanol (MeOH, 20 ml) containing ethanolamine (0.15 mL). The suspension was warmed to 50° C. for a few minutes, giving a virtually clear yellow solution which was filtered and concentrated to 5 mL when crystallization readily began. Diethyl ether (Et₂O, 5 mL) was added and the mixture was cooled to 0° C. to complete crystallization. Filtration, washing with Et₂O containing a little MeOH, afforded the title salt (0.49 g, 75%) as a yellow crystalline solid; mp 158-160° C. (dec.); Found: C, 44.1; H, 4.2; N, 17.35; S, 9.8.

C₁₂H₁₄N₄O₅S requires C, 44.2; H, 4.3; N, 17.2; S, 9.8%; 1H NMR [400 MHz, (CD₃)₂SO] 2.86 (2H, t, CH₂CH₂), 3.57 (2H, t, CH₂CH₂), 5.20 (1H, br s, OH), 6.81 (2H, m, ArH), 7.32 (1H, m, ArH), 7.67 (3H, br s, NH₃⁺), 7.91 (1H, m, ArH), 8.51 (1H, s, 4′-H) and 14.71 (1H, br s, NH); 13C NMR [100 MHz, (CD₃)₂SO] 41.6, 57.9, 117.5, 118.2, 119.9, 130.1, 133.4, 137.9, 145.9, 161.3, 171.6 and 172.2.; m/z (-ye ion electrospray mode) 264 [(M-H)^{□}]. Found: m/z, 264.0092. C₁₀H₆N₃O₅S requires m/z, 264.0085.

### Example 14

RM-5071 is a prodrug of Tizoxanide (TIZ, also known as desacetyl-nitazoxanide or desacetyl-NTZA), the active metabolite of Nitazoxanide (NTZA), an antiprotozoal drug approved by the FDA (Alinia) for the treatment of Cryptosporidium parvum or Giardia lamblia in children (oral suspension) and adults (tablets).

RM-5071 is an organic salt composed of two ionic moieties: tizoxanide alkoxide and ethanolammonium. The chemical structure of ethanolamonium tizoxanide alkoxide, hereafter called RM-5071, is presented below. The laboratory scale synthesis of RM-5071 is essentially a two-step synthesis using NTZA as a starting material. The first step is the removal of the acetyl group of the NTZA by dissolving in warm (70° C.) dimethylformamide (DMF) in the presence of hydrochloric acid (HCl) followed by neutralization by sodium hydroxide (NaOH) at room temperature. The resulting product is filtered prior to the next step. The second step requires dispersion of the filtered powder in methanol, followed by cooling the suspension, while slowly adding ethanolamine to form the salt. The solid is filtered, washed, and dried in a Rotavapor. The final product is a yellow powder identified as RM-5071.

Chemical Structure of RM-5071 (hydroxyethylammonium tizoxanide alkoxide).

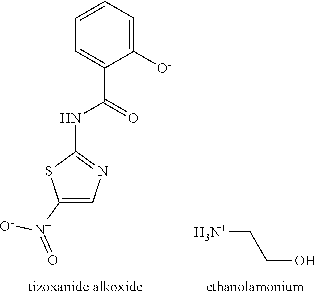

This report gathers physical and chemical characterization data generated to date of the synthetic product described above. The purpose of the physical characterization is to obtain information on characteristics such as melting point, particle size distribution, crystallinity, morphology, and performance under thermal stimuli. The chemical characterization data provide information on the solubility, acid-base properties, chemical functionalities, molecular mass, and spectral characteristics, to gain knowledge about the molecule's chemical behavior.

| TERM | DEFINITION |
|---|---|
| ACN | Acetonitrile |
| API | Active Pharmaceutical Ingredient |
| ATR | Attenuated Total Reflectance |
| DAD | Diode Array Detector |
| DMF | Dimethylformamide |
| DMSO | dimethyl sulfoxide |
| DSC | Differential Scanning Calorimetry |
| EDS | Energy Dispersive Spectroscopy |
| ESI | Electrospray Ionization |
| FDA | Food and Drug Administration |
| FT-IR | Fourier Transform- Infrared Spectroscopy |
| H-NMR | Proton Nuclear Magnetic Resonance |
| HPLC | High Performance Liquid Chromatography |
| LOD | Loss on Drying |
| MCC | Materials Characterization Center |
| MS | Mass Spectrometry |
| NTZA | Nitazoxanide |
| PSD | Particle Size Distribution |
| PTL | Particle Technology Laboratory |
| RT | Room Temperature |
| SEI | Secondary Electron Image |
| SEM | Scanning Electron Microscopy |
| TGA | Thermogravimetric Analysis |
| TIZ | Tizoxanide |
| USP | United States Pharmacopeia |
| UV | Ultraviolet-Visible |
| XRD | X-ray diffraction analysis |

### 1. Discussion

This section describes the analytical tests performed, data, and results from the various physical and chemical characterization techniques listed below:

Physical Characterization
Visual Inspection
Particle Morphology by Scanning Electron Microscopy (SEM)
Particle size analysis by Laser Diffraction calorimetric transitions monitored by Thermogravimetric Analysis (TGA) and Differential Scanning calorimetry (DSC)

Chemical Characterization
Solubility
Acid-base Titration
UV-Vis Spectrophotometry (UV)
Proton Nuclear Magnetic Resonance (¹H-NMR)
Electrospray-Mass Spectrometry (ESI-MS)
Fourier Transform-Infrared Spectrophotometry (FT-IR)
X-ray diffraction (XRD)

A sample of RM-5071 and a sample of desacetyl-NTZA (tizoxanide) were submitted for comparison purposes.

### 1.1 Physical Characterization

### 1.1.1. Visual Inspection

A visual inspection of RM-5071 shows a bright yellow powder with fine particles that agglomerate into easily disturbed lumps. In comparison, a sample of desacetyl-NTZA shows a loose bone-white powder with fine particles.

### 1.1.2. Scanning Electron Microcopy

Scanning Electron Microscopy (SEM) is an analytical technique used to obtain a magnified view of the sample morphology. This is achieved by focusing an electron beam on the sample, controlling the accelerating voltage and therefore, the penetration depth and kinetic energy of incident electrons, to acquire a signal of both backscattered and secondary electrons. The Energy Dispersive Spectroscopy (EDS) system is used, in combination with the SEM system, to obtain the elemental composition of the samples.

### 1.1.2.1 Materials and Equipment

The equipment used for the SEM/EDS analysis by the MCC was JEOL 6480 LV Scanning Electron Microscope equipped with an EDAX X-ray Fluorescence Detecting Unit.

### 1.1.2..2 Procedure

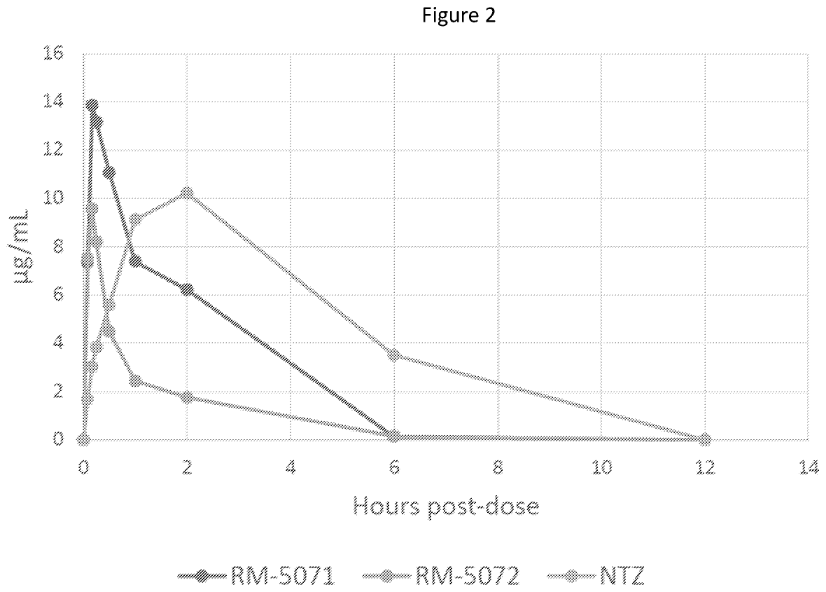

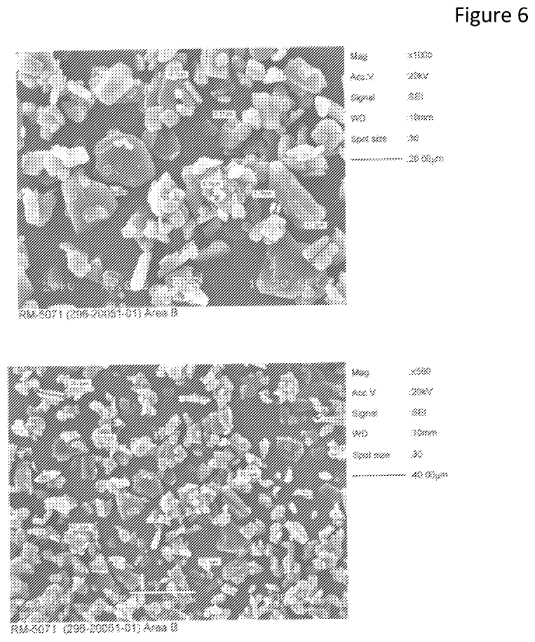

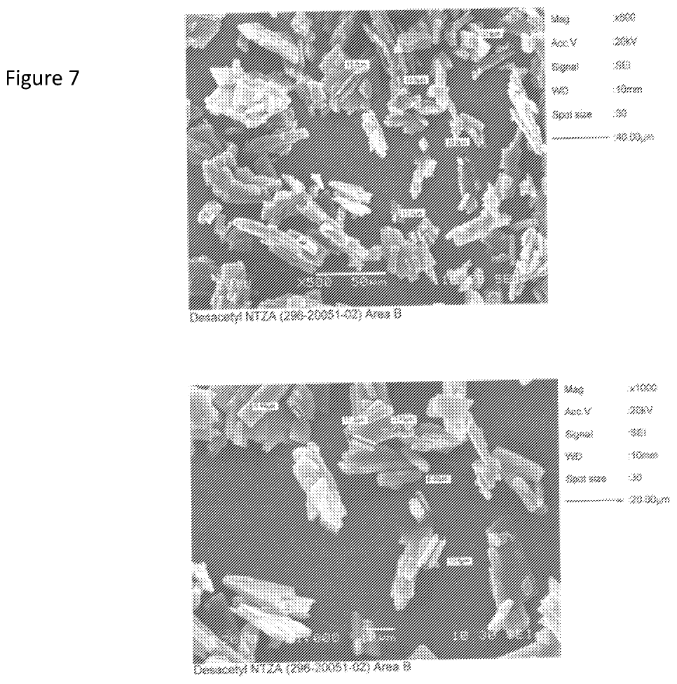

The microscopy was performed by the Material Characterization Center (MCC). Two samples were submitted to MCC for characterization and comparison to the Materials Characterization Center, San Juan, PR. The materials were isolated with the aid of a spatula, and mounted onto double-sided carbon tape previously adhered to aluminum stubs. Backscatter Electron Images (BEI micrographs) were obtained at 1500× magnification. Particle size analyses were performed by gold coating the sample with a ˜30 nm thin film. Both analyses were performed in high vacuum at 20 kV. Secondary Electron Images (SEI) of the samples were obtained between 500× and 1000× magnifications. FIGS. 2 and 3 show the electronic microcopy images for both RM 5071 and desacetyl-NTZA at two different magnifications. As shown on FIG. 6, the RM-5071 particles display a morphology seemingly formed by layers and/or steps. Most of the single particle shapes are elongated; nonetheless, agglomerates can be seen on both areas examined with particles of irregular shapes and round edges. In contrast, the desacetyl-NTZA SEM images in FIG. 7, shows elongated particles with sharp edges.

Comparing visually the SEM images from RM-5071 (FIG. 6) and desacetyl-NTZA (FIG. 7) at the same magnification, it seems that RM-5071 contains particles slightly smaller in size than desacetyl-NTZA. Moreover, a preliminary analysis of particle size was performed by the MCC using the information from the SEM images by measuring a sample of 10 particles per spot. The RM-5071 sample showed particle dimensions approximately from 4.16 to 26.30 μm, with an average of 13 μm. Desacetyl-NTZA showed particle dimensions approximately from 3.45 to 34.30 μm, with an average of 15.1 μm. These results are presented in Table 5. In addition, EDS results were similar in terms of elemental composition of both, RM-5071 and desacetyl- NTZA: Carbon as major element; Oxygen and Sulfur as moderate element; and Nitrogen as minor element. Moreover, traces of aluminum are present in both RM-5071 and desacetyl-NTZA samples due to the aluminum stubs used to hold the carbon tape on top of the sample stage to introduce the sample into the microscope.

TABLE 5
Summary of sample approximate dimensions
measured using SEM images.

| | | Particle Size Range | Average Sample |
|---|---|---|---|
| Sample | Area | (μm) | Particle Size (μm) |
| RM-5071 | A | 4.75-22.60 | 13.08 |
| | B | 4.16-26.30 | |
| Desacetyl-NTZA | A | 5.73-34.30 | 15.08 |

### 1.1.3. Particle Size Distribution Analysis

Laser diffraction is used as a particle sizing method in the range of 0.5 to 1000 microns. It works on the principle that when a beam of light (a laser) is scattered by a group of particles, the angle of light scattering is inversely proportional to particle size.

### 1.1.3.1. Method

Suitable methods for particle size distribution (PSD) for RM-5071 sample using laser diffraction were developed based on the guidelines established in ISO 13320-2009: Particle

Size Analysis-Laser diffraction methods, and USP <429> Light Diffraction Measurement of Particle Size.

### 1.1.3.2. Materials and Equipment

The equipment used for the particle size distribution analysis was Malvern Mastersizer 3000. Dry dispersion was performed using the venturi disperser. For the liquid dispersion, particle analysis sample preparation was performed by dispersing the solid in 2% lecithin in IPG (Isopar G, an isoparaffinic hydrocarbon) and sonicating for 15 seconds using an Elmasonic S ultrasonic bath.

### 1.1.3.3. Procedure

The purpose of this method development stage 1 study is to evaluate sample preparation conditions and instrument settings for particle size analysis of RM-5071 by laser diffraction.

### 1.1.3.4. Results

The RM-5071 sample was first observed under a light microscope to determine the general particle size and shape before moving forward with the evaluation. The particles were observed to be irregularly shaped with primary particles typically <40 μm. Soft to semi-robust agglomerates are visible in the powder at the millimeter size range. These agglomerates can be fairly easily dispersed with pressure, e.g. pressing on the agglomerate with the tip of a spatula.

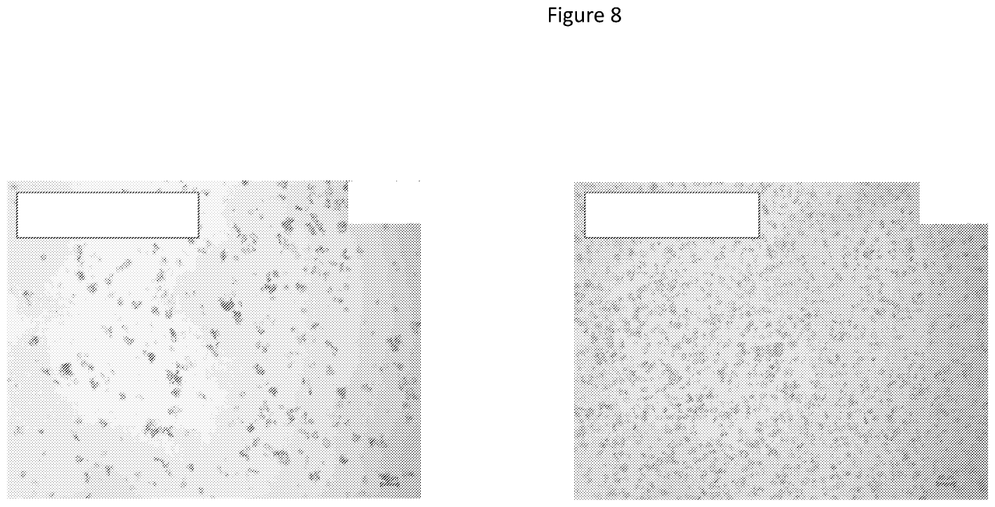

Liquid dispersion particles size distribution analysis was conducted using default settings varying dispersants and carriers to determine an appropriate liquid dispersion. The sample material required some dispersion energy to fully disperse to primary particles. The preparation was analyzed using default settings after sonicating for 15 s. FIG. 8 exhibits digital images of the sample preparation dispersion before and after 15 seconds of sonication, the agglomerates are dispersed after sonication. The preparation using 2% lecithin in IPG (Isopar G, an isoparaffinic hydrocarbon) as the carrier and dispersant appeared to produce the most uniform dispersion by microscopic observation. These conditions were used for comparative analysis to the dry dispersion analysis.

For dry dispersion particle size distribution analysis, a pressure titration was conducted to evaluate the effect of pressure on the RM-5071 particles. The particle size results from this analysis have been summarized in Table 3. Note that the results of the PSD analysis by Laser Diffraction confirm the preliminary observations via SEM in that the individual particles approximate sizes are between 3 and 40 μm and also that agglomerates of primary particles are also observed.

TABLE 6
Summary of the pressure titration study
and liquid dispersion on RM-5071 sample.

| RM-5071 | Cumulative volume % less than Indicated size (μm) | | |
|---|---|---|---|
| Pressure | Dv (10) | Dv (50) | Dv(90) |
| 0.0 bar | 8.07 | 25.4 | 1500 |
| 1 bar | 4.42 | 10.5 | 254 |
| 2 bar | 3.30 | 8.48 | 21.1 |
| 3 bar | 2.65 | 7.56 | 17.9 |
| 4 bar | 2.49 | 7.33 | 19.9 |
| Liquid | 3.73 | 11.0 | 21.4 |
| analysis | | | |

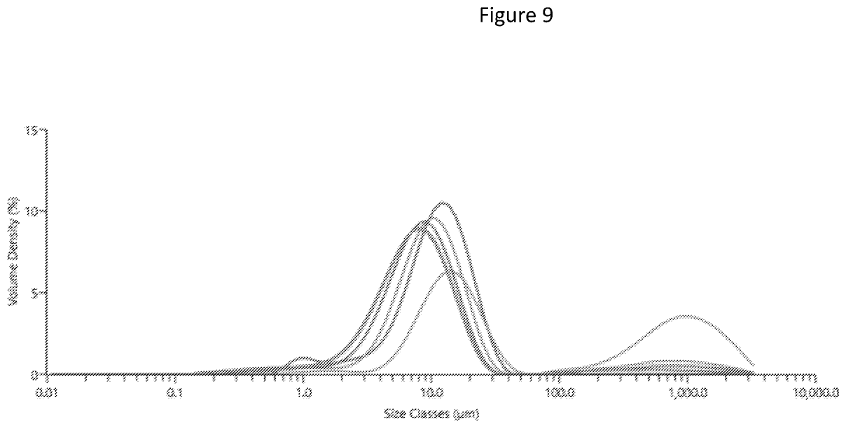

An overlay of the distributions produced at each pressure in comparison to the average liquid distribution has been provided in FIG. 9. The pressure which produces results most similar to the wet dispersion (shown in red) is the optimal pressure to select for the analysis, which is 1 bar, shown in light blue in FIG. 9. Particle size distribution results reveal that the agglomerates present in the sample are not fully dispersed via the attempted dry dispersion settings, due to the observation of a small population of coarse particles at sizes between 100-1200 μm. Additionally, the primary peak of the distribution shifts to smaller particle size as the pressure is increased which is expected as higher pressure might cause primary particle breakage. At the maximum pressure (4 bar), there is still a small amount of coarse particles present in the dry dispersion analysis which indicates that even at this higher energy setting, there still a small amount of agglomerates observed.

The fed rate and hopper height settings were initially selected to allow for stable and complete flow of the sample, but at the conclusion of the pressure titration analyses it became clear that the initially selected settings were insufficient to fully feed the powder into the instrument consistently. The non-ideal sample flow may have been contributing to the incomplete dispersal of the agglomerates and therefore the instrument settings were adjusted to improve flow of sample. The pressure titration with the analysis at 4-bar using the new feed rate (65%) and hopper height (1.5 mm) the particle size distribution remained similar despite the improved sample flow.

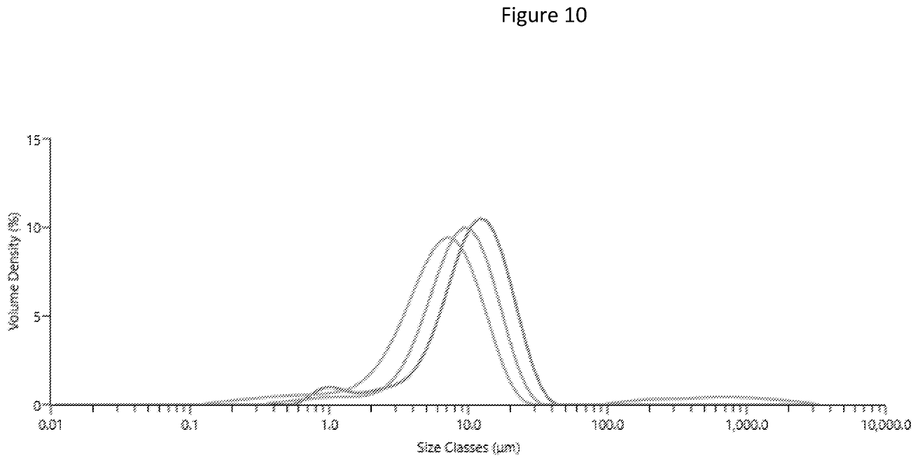

The agglomerates in the aliquot for analysis were dispersed using a spatula prior to addition to the instrument to aid in the dispersion. This successfully resulted in analysis with no coarse peak present (FIG. 10), however, the resulting peak is shifted to finer particle size compared to the liquid analysis. Based on these analysis, preliminary factors were selected for a method development as presented on Table 4 which will require further method development to optimize but at this time is sufficient to characterize the material.

TABLE 7
Preliminary proposed factors by PTL for particle analysis

| Factor | Method setting | Range (±) |
|---|---|---|
| Sample mass (g) | 0.30 | 0.06 |
| Feed rate (%) | 65 | 10 |
| Hopper Height (mm) | 1.5 | 0.25 |

### 1.1.4. Melting Point

Substances exhibit a melting transition, spanning the temperatures at which the first detectable change of liquid phase is detected to the temperature at which no solid phase is apparent. The transition may appear instantaneous for a highly pure material, but usually a range is observed from the beginning to the end of the process. Factors influencing this transition may include the sample size, the particle size, the efficiency of heat diffusion within the sample, and the heating rate, among other variables, that are controlled by procedure instructions.

### 1.1.4.1. Method

The melting point was measured according to the standard operating procedure BEL-SOP-000152 titled “Melting Point Apparatus BÜCHI B-540”.

### 1.1.4.2. Materials and Equipment

The equipment used for the melting point analysis was Büchi B-540.

### 1.1.4.2. Procedure

The quantity of RM-5071 and desacetyl-NTZA equivalent to a height of 4-6 mm in a melting point capillary tube was inserted in the Büchi B-540.

### 1.1.4.4. Results

Differences on melting point for organic molecules with similar structures are useful to distinguish identity, both RM-5071 and desacetyl-NTZA are solid at room temperature. The melting point of RM-5071 and desacetyl-NTZA were measured, results are shown in Table .

TABLE 8
Results for meting point for RM-5071 and desacetyl-NTZA.

| | Desacetyl- |
|---|---|
| RM-5071 | NTZA |
| 146-148° C. | 240-250° C. |

The results show a different melting point temperature for RM-5071 (146-148° C.) as compared to its precursor desacetyl-NTZA (240-250° C.). This difference is a useful physical property identifier of RM-5071 as a new chemical entity. It provides a means to differentiate RM-5071 from desacetyl-NTZA during the synthetic process.

### 1.1.5. Loss on Drying

Loss on drying (LOD) is a test method to determine the moisture content of a sample, although occasionally it may refer to the loss of any volatile matter from the sample.

### 1.1.5.1. Materials and Equipment

The measurement of LOD was performed on a non qualified equipment, the heating was executed on a Binder vacuum oven with an accuracy +/−0.1° C., equipped with a vacuum pump, Vacuubrand MD4C (Pmin=1.5 mbar). The mass of the sample was measured using an analytical balance Mettler AG285 (d=0.01 mg).

### 1.1.5.2. Procedure

The RM-5071 sample was gently pressed with a spatula to crush any agglomerated particles before weighing out the test specimen. The sample was put in a weight bottle to measure its mass by difference before and after being heated at 60° C. and for the 2 hrs in a vacuum oven. Drying continued until two consecutive weighing do not differ by more than 0.50 mg per g of substance taken, the second weighing following an additional hour of drying. This procedure is performed in accordance to USP <741> Loss on Drying.

### 1.1.5.3. Results

The LOD measurement for RM-5071 according USP method is 0.2%. The sample weight was stable after the second drying round. A low loss on drying percent suggest that the RM-5071 solid does not have volatile substances adsorbed that could have remained from synthetic process. This result is in agreement with the data obtained from the thermogramivetric analysis (TGA) discussed in the next section, where RM-5071 does not exhibit any significant weight loss up to 100° C.

### 1.1.6. Calorimetric Analysis

Differential scanning calorimetry (DSC) is a thermo-analytical technique in which the difference in the amount of heat required to increase the temperature of a sample versus a reference are measured as a function of temperature. The reference sample should have a well-defined heat capacity over the range of temperatures to be scanned. One application of DSC is studying phase transitions, such as melting, glass transitions, and/or exothermic decompositions. Such measurements provide qualitative and quantitative information about physical and chemical changes of a molecule.

Thermogravimetric analysis (TGA) is an analytical technique used to determine the thermal stability of a material and its fraction of volatile components by monitoring the weight change that occurs as a substance is heated. As many weight loss curves look similar, the curves may require transformation before results may be interpreted. A derivative weight loss curve can be used to tell the point at which weight loss is most apparent. The analysis is normally carried out in air or in an inert atmosphere such as nitrogen.

### 1.1.6.1. Materials and Equipment

The equipment used for the DSC and TGA was TA Instruments DSC Q2000 and TA TGA Q500 instrument with a TA universal Analysis 2000 program, respectively.

### 1.1.6.2. Procedure

The calorimetric analysis was performed by the MCC. For the DSC analysis, amounts between 2.34 to 2.46 mg of the samples RM-5071 and desacetyl-NTZA were placed individually in aluminum hermetic pans and covered with an aluminum lid. The temperature method was: step 1: hold for 10 min at 25° C., and step 2: heat from 25° C. to 400 at 10.00 ° C./min. An additional DSC experiment was performed to determine further crystallinity behavior by performing a heat-cool-heat cycling experiment to RM-5071. Approximately 1.69 mg of the RM-5071sample was placed in an aluminum hermetic pan and covered with an aluminum lid. The temperature method was: step 1: hold for 5 min at 25° C., step 2: heat from 25 to 350° C. at 10.00° C./min., step 3: cool from 350 to 25° C. at 10.00° C./min; and step 4: heat from 25 to 350° C. at 10.00° C./min. Both analyses were carried out under nitrogen.

For the TGA analysis, amounts between 2.0 to 2.6 mg of both RM-5071 and desacetyl-NTZA samples were placed individually in platinum pans. The temperature method was: step 1: equilibrate at 25° C., step 2: heat from 25° C. to 700° C. at 10.00° C./min, step 3: hold for 5 min at 700° C. The analyses were carried out under nitrogen.

### 1.1.6.3. Results

FIG. 11A (left) contains the thermogram of RM-5071 and it shows a first step weight loss of approximately 20% between a temperature range of 85-160° C. and a total weight loss of 77% after three discrete thermal transitions. The first weight loss of approximately 20% is consistent with the hypothesis of the loss of ethanolammonium (average molecular weight=62.09 amu) which is 19.03% of the total weight of the RM-5071 (average molecular weight=326.33 amu). The desacetyl-NTZA showed a first step weight loss of 36% in the temperature range of 199-280° C. and a total weight loss of ˜67% after two transitions. Based on these results, the thermal properties of RM-5071 and desacetyl- NTZA are significantly different, specifically on the number and temperatures of the thermal transitions that both molecules undergo. Therefore, the presence of ethanolammonium results in a different thermal stability behavior between the desacetyl-NTZA precursor and its ethanolammonium salt.

TABLE 9
Summary of results of the thermogravimetric analysis.

| | | |
|---|---|---|
| | | Desacetyl- |
| Results | RM-5071 | NTZA |

| | | | |
|---|---|---|---|
| First step | Weight loss (%) | 19.51 | 36.38 |
| | Temperature (° C.) | 85-160 | 199-280 |
| Second step | Weight loss (%) | 30.93 | 30.96 |
| | Temperature(° C.) | 160-254 | 280-700 |
| Third step | Weight loss (%) | 26.62 | N/A |
| | Temperature(° C.) | 254-700 | N/A |

The DSC thermogram of RM-5071 in FIG. 12 exhibits one peak transition at 163° C. This transition temperature is lower than the transition temperature of 286° C. observed in the DSC thermogram for desacetyl-NTZA. Both molecules show exothermic transitions. The heat-cool-heat cycling experiment results for RM-5071 show that the transition at 163° C. is irreversible, thus the transition could be due to the loss of ethanolammonium, degradation of the molecule or both. In conclusion, the precursor, desacetyl-NTZA is more thermally stable than its ethanolammonium salt.

### 1.2. Chemical Characterization

### 1.2.1. Solubility

According to the USP, solubility is the capacity of the solvent to dissolve a solute and is defined in units of concentration. The solubility of a solid is a function of polarity and temperature. The apparent solubility is the empirically determined solubility of a solute in a solvent where insufficient time is allowed for the system to reach equilibrium. Whereas, equilibrium solubility is the solubility limit at thermodynamic equilibrium, to which a solute may be uniformly dissolved into a solvent when an excess solid is present. Understanding the solubility of RM-5071 may be important for the development of validated analytical methods for quantitation of the analyte and its related substances. In addition, the knowledge derived from solubility studies is fundamental for the eventual drug product formulation.

### 1.2.1.1. Methods

The apparent solubility of RM-5071 was tested using several solvents, specifically: DMF, DMSO, acetonitrile (ACN), water, ACN/water mixture, methanol, isopropanol, and ethanol. United States Pharmacopeia (USP) classify the solubility regardless of the solvent used, just following defined criteria summarized in Table 10. The solute was initially dissolved in each of the solvents or solvent system at a lmg/mL concentration. If solute remained undissolved, a fresh solution of lower concentration was subsequently prepared until the solid was observed completely dissolved. This concentration limit was estimated as the apparent solubility.

TABLE 10
USP Description of Solubility from the General
Notices and Requirements Chapter, Section 5.30

| Descriptive | Parts per solute (mg) per |
|---|---|
| term | parts of solvent (mL) |
| Very soluble | Higher than 1 mg/mL |
| Freely soluble | Between 1 mg/mL and 0.1 mg/mL |
| Soluble | Between 0.1 mg/mL and 0.033 mg/mL |
| Sparingly | Between 0.033 mg/mL and 0.01 mg/mL |
| soluble | |
| Slightly soluble | Between 0.01 mg/mL and 0.001 mg/mL |
| Very slightly | Between 0.001 mg/mL and 0.0001 mg/mL |
| soluble | |
| Practically | Equal or less than 0.0001 mg/mL |
| insoluble, or | |
| Insoluble | |

### 1.2.1.2. Materials and Equipment

All chemical substances used on solubility test are specified on Table 11.

TABLE 11
Materials used for RM-5071 solubility and
UV-Vis spectrophotometry experiments

| Name | Supplier |
|---|---|
| Dimetylformamide | Sigma |
| | Aldrich |
| Dimethyl | Sigma |
| Sulfoxide | Aldrich |
| Acetonitrile | Sigma |
| | Aldrich |
| | Fisher |
| Water | Milli Q |
| Methanol | Sigma |
| | Aldrich |
| Ethanol | Sigma |
| | Aldrich |
| Isopropanol | Sigma |
| | Aldrich |

### 1.2.1.3. Procedure

The solutions of RM-5071 were prepared, dissolving 10 mg in increasing solvent volumes ranging from 10 mL, 100 mL, to 1000 mL in each of the solvents. According to the visual inspection and observations the apparent solubility of RM-5071 was classified according the 10, as defined by the USP.

### 1.2.1.4. Results

The solubility studies of RM-5071 were performed in several solvents as specified on Table 11 at room temperature. For the purpose of this chemical characterization report, the terms specified on Table 10 were used to describe the solubility of RM 5071 in different solvents on Table 12

TABLE 12
Summary of RM-5071 solubility and appearance of resulting solutions.

| | Apparent Solubility of | |
|---|---|---|
| Solvent | RM-5071 | Observations |
| DMF | Very soluble | Readily dissolves in DMF. |
| DMSO | Very soluble | Readily dissolves in DMSO. |
| Acetonitrile | Soluble | At 1 mg/mL RM-5071 did not completely |
| | (≤0.033 mg/mL) | dissolve in ACN. Small lumps remained. |
| | | At 0.025 mg/mL the solute dissolved. |
| Acetonitrile:water | Very soluble | Completely dissolves in the mix ACN/Water |
| (35:65) | | (35:65) |
| Methanol | Soluble | Initially, at 1 mg/mL RM-5071 did not dissolve |
| | (≤0.033 mg/mL) | completely and small lumps remained. After |
| | | 3 hrs hours, the lumps dissolved. |
| Ethanol | Soluble | Initially, at 1 mg/mL RM-5071 did not dissolve |
| | (≤0.033 mg/mL) | completely and small lumps remained. After |
| | | 3 hrs hours, the lumps dissolved. |
| Isopropanol | Soluble | At 1 mg/mL RM-5071 did not dissolve |
| | (<0.033 mg/mL) | completely and small lumps remained. |
| Water | Less than slightly soluble | At 1 mg/mL RM-5071 did not dissolve |
| | | completely, and the solution was cloudy. |
| | | After 4 hrs, the solution changes color from |
| | | yellow to orange but remained cloudy. |

RM4-5071 did not dissolve completely in water at concentrations as low as 0.005 mg/mL. Lower concentrations were not attempted due to practical limitations (ability to weigh less than 10 mg and unavailability of flasks larger than 1L). Regardless, there is some amount of solute dissolved in water as evidenced by the yellow color of the solutions and the observation of a UV-vis spectrum in the water supernatant which is discussed further in section 5.2.3. The RM-5071 solution prepared at a concentration of 1 mg/mL was light yellow and turned orange with time (hours), which can be an indication of possible degradation or instability of the molecule in water.

RM-5071 is very soluble (>1 mg/mL) in a 65:35 ACN:water mixture. This finding is somewhat surprising since the solubility in pure in acetonitrile and water are much lower 0.033mg/mL and <0.005 mg/mL, respectively). Nevertheless, according to Qiu, et al. Organic Process Research & Development 2019, 23 (7), 1343-1351 for some charged solutes (APIs) studied, a small amount of water mixed with organic solvents increases the API solubility as compared to the solubility of the solute in the individual pure solvents. This behavior is known as synergistic solvation effect (a.k.a parabolic solubility). The same effect was observed for RM-5071 in mixtures of 25:75 and 50:50 water:alcohols (isopropanol and ethanol).

### 1.2.2. Acid-Base Titration Curves

Direct titration is the treatment of a soluble substance (titrate), contained in solution in a suitable vessel (titrate), with an appropriate standardized solution (titrant), the endpoint of the reaction being determined instrumentally or visually with the aid of a suitable indicator. When a series of pH measurements as a function of volume (mL) of titrant added to the titrate are plotted for an acid-base titration, a sigmoidal curve results with a rapidly changing portion in the vicinity of the equivalence point. Chemical information such as pKa (acid/base equilibrium constant) can be derived from such curve.

### 1.2.2.1 Materials and Equipment

The pH meter used for the measurement during the addition of the titrant was a Mettler-Toledo Seven Excellence. Table 13 contains all the materials used during the experiment.

TABLE 13
Materials used on the potentiometric curves on RM-5071.

| Name | Supplier |
|---|---|
| Water | Milli Q |
| HCl | Fisher |
| | Scientific |
| NaOH | Fisher |
| | Scientific |

### 1.2.2.2. Procedure

The acid titration was performed by adding 20 mL of a 1 mg/mL RM-5071 solution poured onto a beaker (titrate), the pH of the solution was measured while using a solution of 0.01N HCl as the titrant. The alkaline titration was performed by adding 20 mL of a 1 mg/mL RM-5071 solution were poured onto a beaker, the pH of the solution was measured while adding a solution 0.02 N NaOH as the titrant. The solutions had agitation with a magnetic stirrer during titration and it was stopped during pH measurements.

### 1.2.2.3. Results

Changes in color and solubility of 20 mL RM-5071 solution as an acidic titration progresses by the addition 0.01 N HCl to the RM-5071 solution were observed. Initially, a 1 mg/mL RM-5071 solution has a pH of 9.44. As acid is titrated to the beaker, the pH decreases as expected. In addition, as the pH decreases the solution becomes increasingly cloudy and a solid precipitates. At pH 3.80 the physical appearance of the beaker contents is a beige powder suspended in a faint yellow solution. The appearance of the suspended and settled powder is similar to the desacetyl-NTZA, suggesting that the phenolic hydroxyl group in the molecule has been protonated, thus reducing its polarity and consequently also reducing its solubility in water and precipitating out of solution.

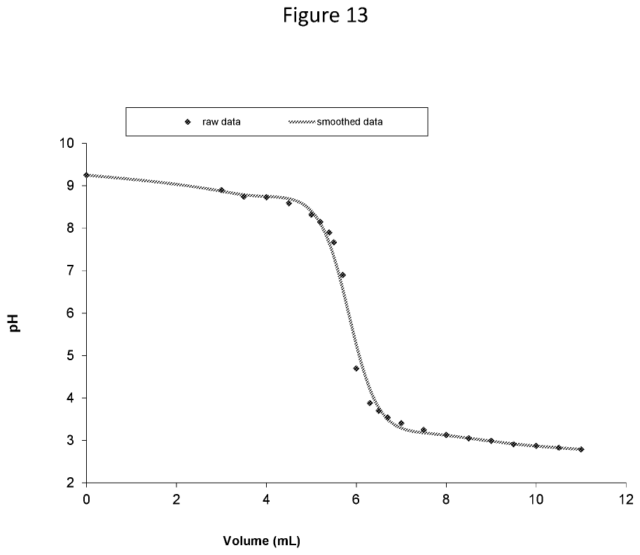

The titration curve on FIG. 13 shows the inflection point of the acid-base titration of RM-5071. The inflection point was calculated using the second derivative of the experimental curve, the volume for the equivalence point is 5.83 mL of 0.01 N HCl, at a pH =5.99. With this information and the RM-5071 mass, the stoichiometry of the acid-base reaction calculated, resulted in one proton per RM-5071 molecule. The point at half the volume of the inflection point is 2.92 mL, resulting in a pKa of 8.8 for RM-5071. The pKa of a phenolic proton is 9.9 as per Vollhardt, K. P. C.; Schore, N. E., Organic Chemistry; Palgrave Version: Structure and Function. Macmillan International Higher Education: 2014. Thus it is reasonable to conclude that the proton titrated with acid at an estimated pKa of 8.8 is the phenolic proton circled below:

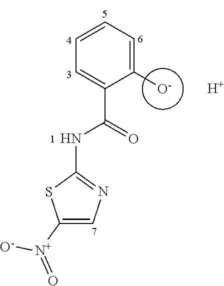

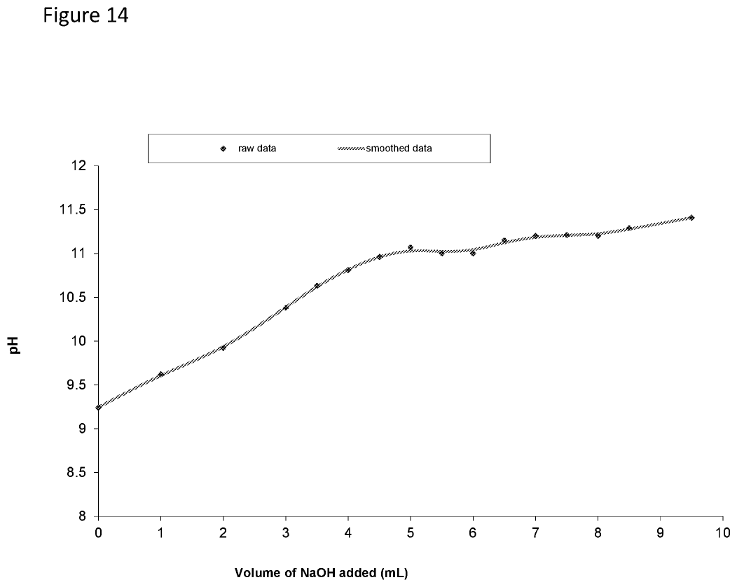

Images of the beaker containing the RM-5071 solution as 0.01N NaOH solution showed the solution gradually changing from a cloudy yellow suspension to a clear orange color as the pH increased. It is observed that RM-5071 increasingly dissolved as the pH increases and achieving full dissolution of the solid at pH=10.63. The titration curve is in FIG. 14 and no clear inflection point can be appreciated, thus no pKa information in the acidic pH range can be derived from the titration curve. One possible but not-limiting explanation of the absence of a clear inflection point in the titration curve could be that the RM-5071 molecule could assume various resonant chemical species after deprotonation of the amide nitrogen. Such explanation may be consistent with the observation of a solution absorption at higher wavelengths (red-shift).

### 1.2.3. UV-Vis Spectrophotometry

The wavelength (λ) of the absorption corresponds to the difference in energy among the ground and excited states. As a molecule absorbs UV-Visible radiative energy, an electron is promoted from an occupied orbital to an unoccupied orbital of greater potential energy. The electrons may undergo several possible transitions of different energies. In the case of RM4-5071 probable transitions may be σ→π\hat{ }* and π→π\hat{ }* , which are characteristic of carbonyl and conjugated double carbon bonds chemical functionalities, respectively. The transitions that result in the absorption of electromagnetic radiation in the UV-Vis region of the spectrum are transitions between electronic energy levels, and is directly related to the wavelength of the absorption.

The intensity of an absorption is related to the concentration of the species in solution. The Beer-Lambert Law defines the relationship between the intensity of visible UV radiation at a specific wavelength (λ), and concentration of the substance present in the analysis. The Beer—Lambert Law, A=εlc, where A is the absorption at λ, ε is the molar absorptivity coefficient at λ,1 is the optic path length and, c is the concentration of the species. The UV-Visible spectroscopic results from characterization experiments performed provide chemical information on two aspects, electronic absorption spectra in various solvents and quantification of the molecule in the supernatant solution based on the absorption at λₘₐₓ in water.

### 1.2.3.1 Materials and Equipment

A list of the materials used during the UV-Vis spectroscopy is provided in Table 11. The equipment used in this study is a Shimadzu UV-1800 Series.

### 1.2.3.2. Procedure

The UV-Vis analysis was performed by preparing a solution of approximately 1.00 mg of RM-5071 in a 1.0 L volumetric flask and filling to volume with solvent. The resulting solution (supernatant if solid was still present) was transferred to a quartz cuvette with standard path length of 10 mm. The UV-Vis spectra of RM-5071 was performed on the following solvents: DMF, DMSO, water, ACN/water, Methanol, Isopropanol and Ethanol. The instrument specific conditions are on Table 14.

TABLE 14
Instrumental settings for the UV-Vis spectroscopic measurements
Measurement Properties

| | |
|---|---|
| Wavelength range | 200.00 to |
| (nm) | 900.00 |
| Scan speed | Slow (0.5 s) |
| Sampling interval | 0.50 nm |
| Auto sampling | Enabled |
| Scan mode | Repeat |

### 2.3.3. Results

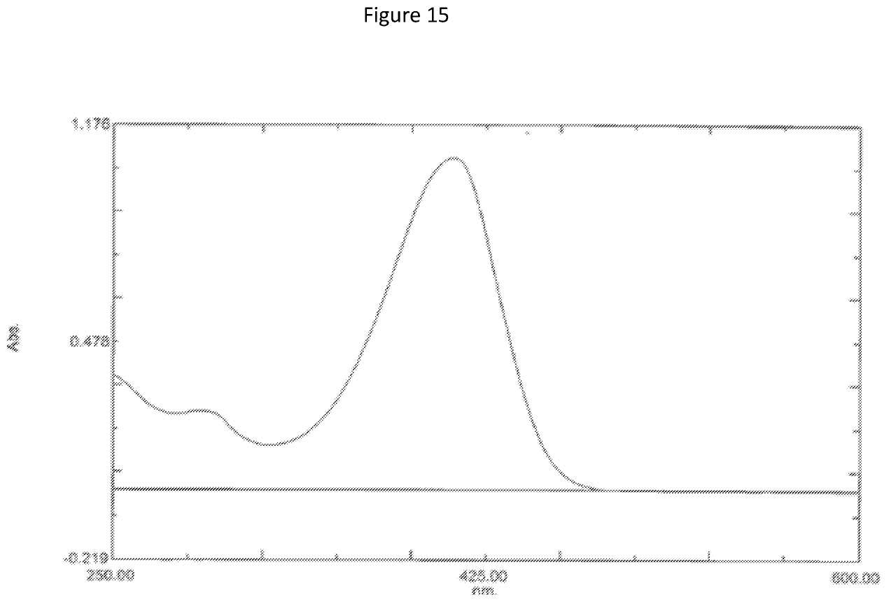

The λₘₐₓ for RM-5071 in different solvents are summarized on Table 12. Compounds that are highly colored (have absorption in the visible region) are likely to contain a long-chain conjugated system or a polycyclic aromatic chromophore, FIG. 15 show a representative UV-Vis absorption spectrum for RM-5071 in methanol, different solvents exhibit a similar spectra with minors shifts according to the λₘₐₓ presented on table 15. Benzenoid compounds may be colored if they have enough conjugating substituents; this is consistent with the visual observation of the solutions presented on table 15.

TABLE 15
At a wavelength of absorption near 400 nm, the color of the solution
observed is yellow, the solubility studies of RM-5071 in water.

| Solvent | λₘₐₓ (nm) | Visual observation |
|---|---|---|
| DMF | 430 | Readily dissolves in DMF. Color: yellow. |
| DMSO | 431 | Readily dissolves in DMSO. Color: yellow |
| ACN | 424 | Partially dissolved. |
| | | Supernatant color: yellow |
| Water | 409 | Partially dissolved. |
| | | Supernatant color: yellow. |
| ACN:Water | 416 | Completely dissolves in the |
| | | mix ACN/Water (35:65) |
| | | Color: yellow |
| Methanol | 409 | Partially dissolved. |
| | | Supernatant color: yellow. |

The Beer-Lambert' s Law is used for analyte quantification using the linear relationship between the absorption at a specific λ and the analyte concentration in solution. Solutions of RM-5071 in water were prepared at 0.051, 0.025, 0.010, 0.0075 and 0.0051 mg/mL. Small lumps of the solid remain at the bottom of the flask for these aqueous solutions, thus sonication was used to aid the dissolution of the solid. After 5 min of sonication, the solid particles were no longer observed, except for the 0.05 mg/ml solution. The absorbance of the supernatant of these solutions were measured after settling for 1 hour after preparation. These solutions were later centrifuged and the spectra of the supernatant solution were also obtained. The UV-Vis absorption were measured at λmax=409 nm.

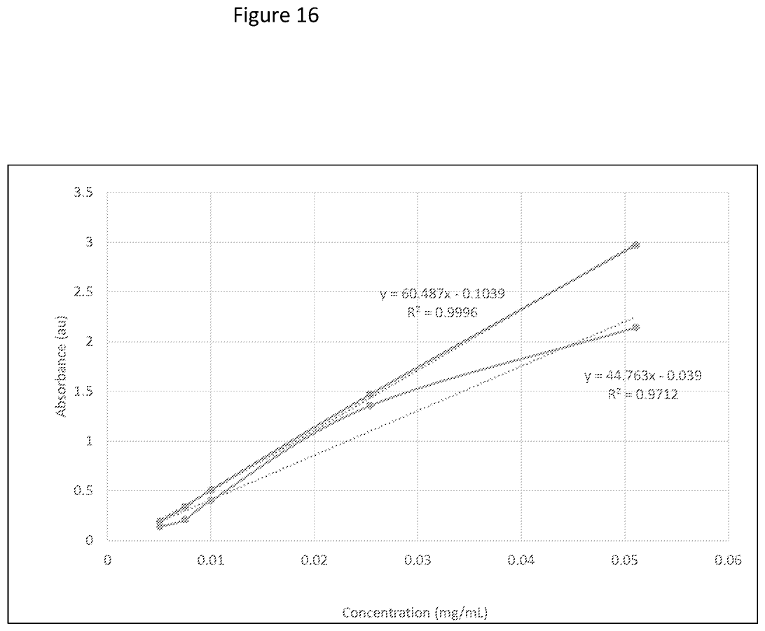

FIG. 16 contains the Absorbance vs. theoretical concentration of RM-5071 in water. The red curve represents the absorbance measurements of the supernatant after settling and the blue line represents the absorbance measurements of the supernatant after centrifugation. The comparison between the two absorbance vs. concentration curves, shows that the r2 (correlation coefficient), is closer to 1 for the blue line (0.99) as compared to the red line (0.97). This is an indication that the RM-5071 concentration reaches equilibrium after centrifugation, because the linearity of this plot is an indication of conformance to Beer Lambert law. Therefore, this is an indication that the centrifugation aided in the dissolution of the solid.

### 1.2.4. Nuclear Magnetic Resonance

Proton Nuclear Magnetic Resonance (¹HNMR) is a technique that can provide chemical structural information of organic molecules. The technique is based on the low energy absorption in radiofrequency range (α=1-5 m) of the proton nucleus within a strong magnetic field. The basis of ¹HNMR is that proton atoms in different chemical environments will have slightly different energetic levels in the presence of the external magnetic fields, resulting in distinct radio frequency energy absorption in their ¹HNMR spectra. The NMR spectra contains information that can be used to derive the functional groups of a molecule based on how shielded the hydrogen atoms are to the surrounding magnetic field. For instance, in the ¹HNMR spectra, it is possible to determine the number of distinct types of hydrogen nuclei and obtain information regarding the nature of the immediate environment surrounding each type. This chemical information may be valuable since it can be used, in combination with the information obtained from other chemical characterization techniques, to deduce and/or confirm the proposed chemical structure of a synthetic molecule. In addition, the integration of the areas under the peaks in the spectra can be used to deduce relative quantification information with respect to molar proportions between the components of the RM-5071 salt.

### 1.2.4.1. Materials and Equipment

NMR Experiments were performed using a 400 MHz Brucker NMR at the Katholieke Universiteit, Leuven, Belgium (KUL)

### 1.2.4.2 Procedure

NMR measurement were performed by dissolving 5 mg sample in 500 μL DMSO-d6. The RM-5071 spectra was acquired with a dedicated NMR method (¹H), with a 400 MHz equipment having a D1=30 s, allowing quantification of the integrals of peaks at 8.5 and 2.8 ppm.

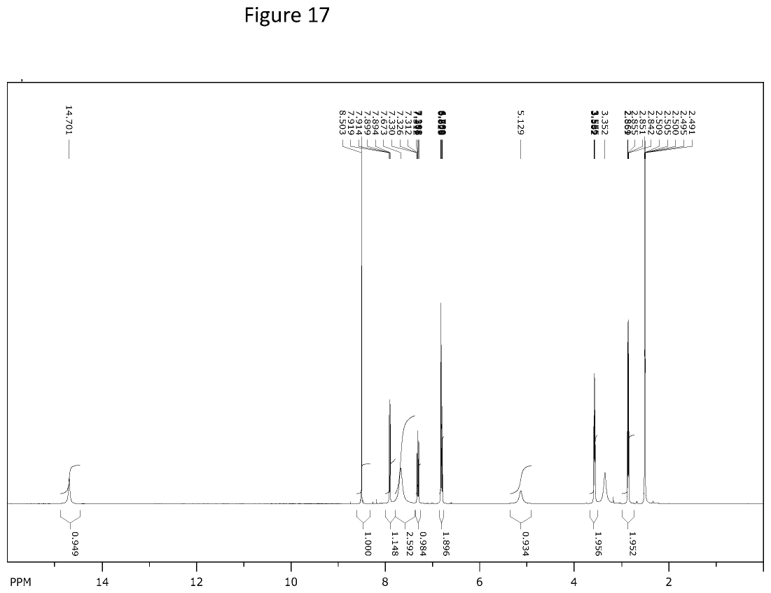

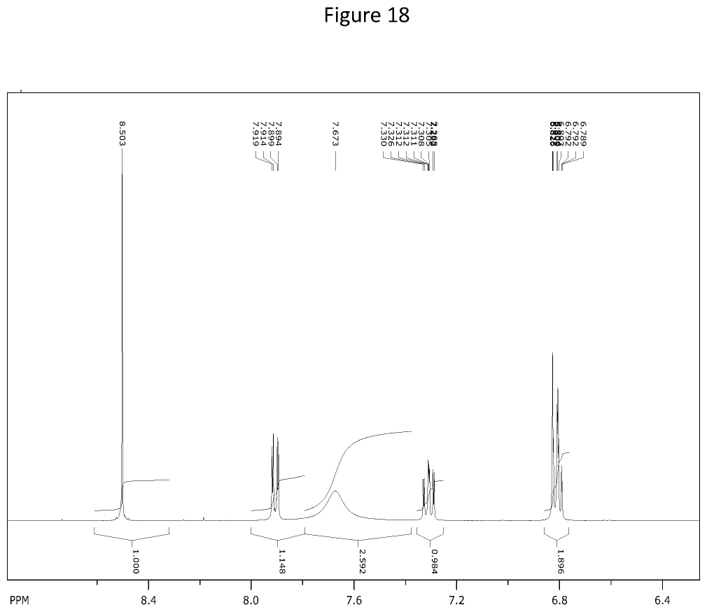

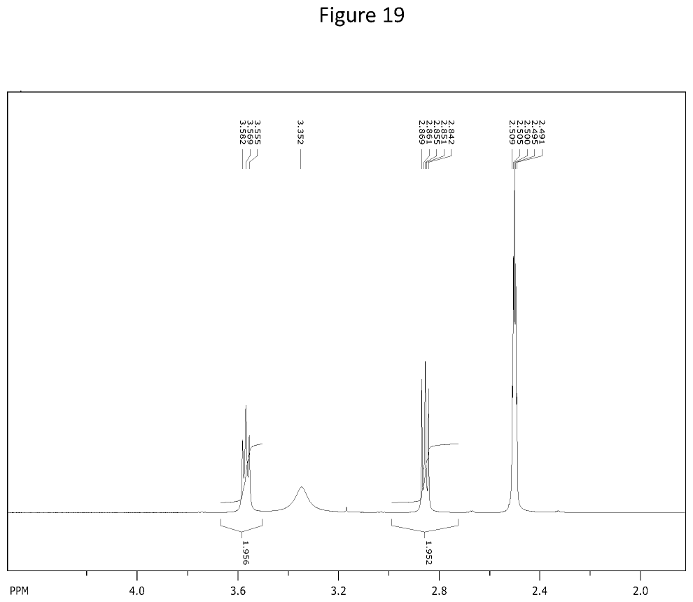

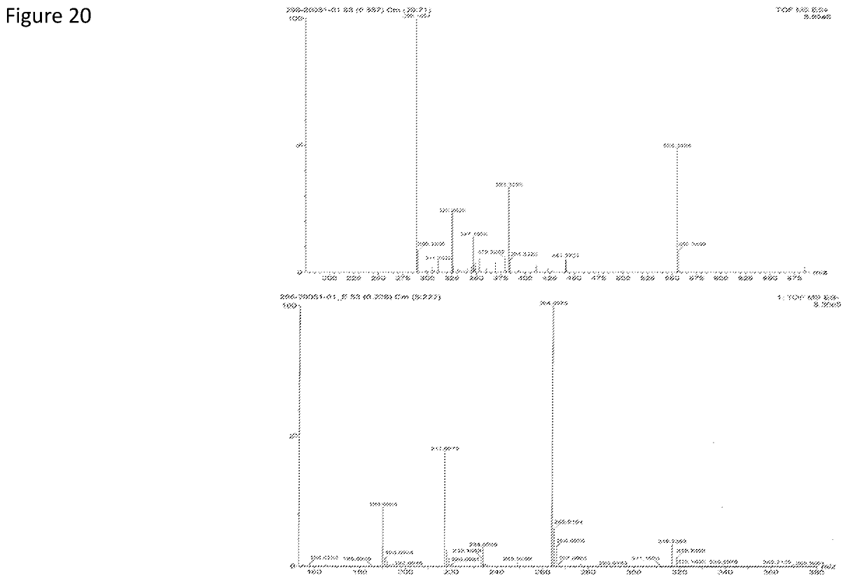

FIG. 17 contains the ¹HNMR spectrum of the RM-5071 molecule and, FIG. 18 and FIG. 19 contain expansions of the various regions of the spectrum to appreciate the details of the peak patterns. The chemical shift (ppm) provides information of the chemical environment surrounding a particular hydrogen atom, the integration of the areas under the peak provides information of how many protons sense the same chemical environment, while the peak splitting pattern provides information about the neighboring protons in the molecule.

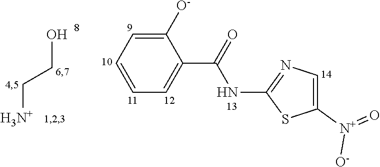

The spectral peak assignment based on the proposed salt molecule structure of RM-5071 containing a total of 14 protons is below.

The peak assignment shows presence of both expected counter ions: ethanolammonium and tizoxanide alkoxide (desacetyl-NTZA). The singlet peak at the highest chemical shift of 17.701 ppm is assigned to the proton (H-13) in the amide functionality in the tizoxanide alkoxide next to electronegative atoms. The following singlet peak at 8.503 ppm belongs to the hydrogen atom in the thiazole ring of desacetyl-NTZA (H-14). The doublet of doublets at 7.8-7.9 ppm range is assigned to the proton of the aromatic proton labeled as H-9 which is the closest proton to the electronegative oxygen atom. The peak at 7.673 ppm is assigned to the three equivalent “interchangeable” H atoms next to the nitrogen in the ethanolammonium ion (H-1,2,3) since the signal integration is 2.6 which corresponds to ˜3 protons and is a “broad” singlet, all characteristics which is are consistent with what would be expected for labile amine protons. The peak with at complex splitting pattern at ˜7.3 ppm is assigned to the aromatic hydrogen at position 10 (H-10). This is because at a first glance it resembles a triplet in which each peak of the triplet peaks has further splitted and appears at higher shifts which is consistent to a hydrogen closer to the phenol oxygen atom with two non-equivalent neighboring hydrogens. The complex splitting pattern of the multiplet at ˜6.8 ppm integrates as the signal from two hydrogens which are assigned to the hydrogens 11 and 12. The singlet at 5.129 ppm is assigned to the alcohol proton of the ethanolammonium. The triplets at 3.5 and 2.8 ppm are assigned to the aliphatic hydrogens in the ethanolammonium ion, H-6,7 closer to the oxygen, and H-4,5 closer to the nitrogen, respectively.

Integration values from ¹HNMR spectra may be not accurate enough to allow a strict quantification. However, the NMR peak integration data can be used to estimate a molar ratio between the ethanolammonium and tizoxanide alkoxide ions in the RM-5071 salt. For example, the area of the peak at 8.50 ppm (H-14 of the thiazole ring) is set a 1.00 (reference). Then, the observed areas of the peaks at ˜2.8 ppm and 2.3 ppm (aliphatic H of ethanolamine) integrate to 1.956 and 1.952, respectively. This is consistent with the expected molar ratio between these two hydrogens if the molar ration between the ions is 1:1.

| | | | | |
|---|---|---|---|---|
| | | | Peak splitting | Coupling |
| H-atom | δ (ppm) | Integration | pattern | constants |

Ethanolammonium

| | | | | |
|---|---|---|---|---|
| (H-1,2,3) N—H₃ | 7.7 | 3H-equivalent | Broad singlet* | N/A |
| (H-4,5) CH₂ | 2.9 | 2H-equivalent | Triplet | ³J_{H4,5-H6,7} = 8 Hz |
| (H-6,7) CH₂ | 3.6 | 2H-equivalent | Triplet | ³J_{H6,7,-H4,5} = 8 Hz |
| (H-8) O—H | 5.1 | 1H | Broad singlet* | N/A |

Tizoxanide Alkoxide

| | | | | |
|---|---|---|---|---|
| (H-9) C—H | 7.9 | 1H | Doublet of doublets | ³J_{H9-H10} = 8 Hz |
| | | | | ⁵J_{H9-H11} = 2 Hz |
| (H10) (H11) C—H | ~6.8 | 2H-not-equivalent | Multiplet | N/A |
| (H-12) C—H | 7.3 | 1H | Triplet of triplets | |
| (H-13) N—H | 14.7 | 1H | Broad singlet* | N/A |
| (H-14) C—H | 8.5 | 1H | Singlet | N/A |

*interchangeable protons

Electrospray ionization (ESI) is a type of ionization technique, where the analyte molecule is brought to the gas phase by the formation of charged liquid droplets for subsequent mass spectrometry (MS) analysis. These charged droplets undergo a process of desolvation as they are introduced in a vacuum that avoids fragmentation of the molecular ion. ESI may be useful in producing ions from large organic molecules because it overcomes the propensity of these molecules to fragment when ionized. The information obtained from an ESI-MS spectrum is useful for characterization purposes because the molecular weight of the intact molecule can be derived from the resulting mass spectrum. The positive ion mass spectra of organic molecules typically correspond to the protonated species ([M+H]⁺, [M+2H]²⁺, etc.) and sodium, potassium or other cation adducts ([M+Na]⁺, [M+K]⁺, etc. The negative ion mass spectra typically consists of the deprotonated species ([M-H]⁻, [M-2H]²⁻, etc.).

### 1.2.5.1. Materials and Equipment

The equipment used on the direct infusion electrospray ionization mass spectrometry (ESI-MS) at the MCC is a research instrument Xevo G2-S QToF (Quadrupole- Time of Flight) with Mass Lynx data acquisition software. The instrument used for the HPLC-DAD-ESI-MS experiments in Landen, Belgium is an Agilent 1100 HPLC with simultaneous diode array detection (DAD) and Electrospray ionization (ESI) with InfinityLab MSD G6100 Quadrupole mass spectrometry (MS) detection fitted with Open Lab data acquisition software. The flow from the HPLC is splitted ⅔ to DAD and ⅓ to ESI-MS.

### 1.2.5.2. Procedure

Sample preparation of approximately 6 mg of RM-5071 were weighted. The RM-5071sample was transferred into 10 mL volumetric flasks and completed to the mark either with dimethyl sulfoxide (DMSO) for Positive-ESI or methanol for Negative ESI. A further dilution for both samples was performed, and then the samples were injected by direct infusion into the system. The instrumental settings and equipment specifications are detailed on Table 16.

Table 16. Instrumental settings for the ESI-MS experiments performed at the MCC

Electrospray Ionization Mass Spectrometer

| Polarity | ESI Positive | ESI Negative |
|---|---|---|
| Capillary Voltage (kV) | 3.0 | 1.7 |
| Sampling Cone (V) | 30 | 20 |
| Desolvatation Temperature (° C.) | 450 | 300 |
| Source Temperature (° C.) | 120 | 120 |
| Desolvation Gas Flow (L/h) | 1000 | 600 |
| Cone Gas Flow (L/h) | 0 | 0 |
| Source Offset (° C.) | 40 | 20 |
| Mass Range (amu) | 100-1000 | 100-1200 |

The procedure in Belgium was to analyze the sample of RM-5071 at a concentration between 0.61 mg/mL in Dimethyl formamide (DMF). The mixture was injected in the Agilent InfinityLab MSD G6100 with the following chromatographic conditions:

TABLE 17
Instrumental settings for the HPLC-DAD-ESI-MS
experiments performed at Romark Belgium

| Parameter | Value | | |
|---|---|---|---|
| Column | Ascentis Express C18, 2.7 μm, 10 cm × 4.6 mm | | |
| | (Sigma Aldrich ref. 53827-U) with column guard | | |
| Gradient | A: 0.1% Formic acid in water (% v/v) | | |
| | B: 0.1% Formic Acid in Acetonitrile (% v/v) | | |
| | Gradient: | | |
| | Time (min) | % A | % B |
| | 0 | 90 | 10 |
| | 1.0 | 90 | 10 |
| | 8.0 | 0 | 100 |
| | 10.0 | 0 | 100 |

| | |
|---|---|
| DAD wavelength | Acquisition range from 190-550 nm |
| Column Temperature | 25° C. |
| Flow | 1.5 mL/min |
| Run time | 10 min |
| MSD scan range | m/z 100-1000 |
| Gas temperature | 300° C. |
| Gas flow | 11 L/min |
| Nebulizer pressure | 15 psi |
| Capillary Voltage | 4000 V |

Chromatograms of absorbance (DAD) and total ion chromatograms (MD) vs. time were generated. Mass spectra of the peak for tizoanide alkoxide as identified with a standard in the absorbance chromatogram was obtained in positive and negative modes.

### 1.2.5.3. Results

A summary of the main ion m/z (mass to charge ratio) signals for positive and negative ionization ESI-MS is shown on Error! Reference source not found. 18. Error! Reference source not found., contains the Positive ESI-MS spectrum for RM-5071 dissolved in DMSO. The negative ion ESI-MS spectra for RM-5071 in methanol is also shown in Error! Reference source not found., where the base peak at m/z 264 is observed and corresponds to the [M-H]⁻¹ ion of tizoxanide alkoxide.

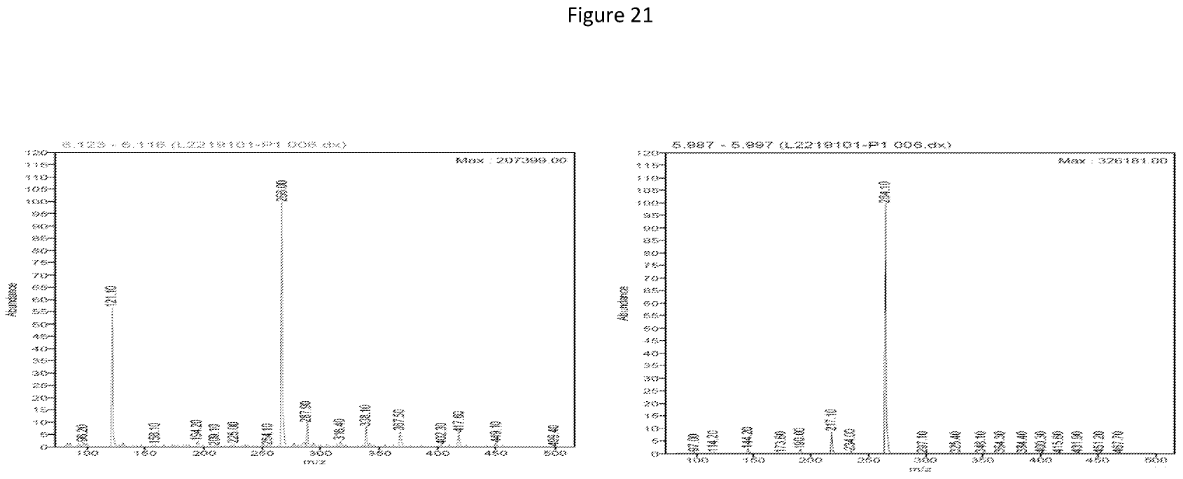

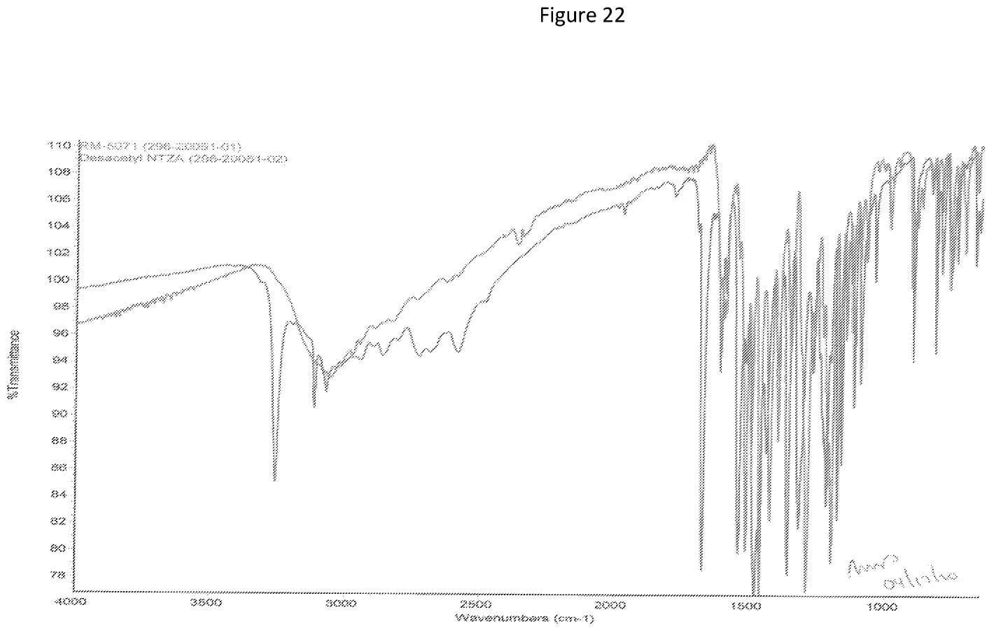

The experiments performed in Belgium after chromatographic separation produced the total ion chromatogram. The electrospray ionization mass spectra of the tizoxanide alkoxide peak from the total ion chromatogram is shown in FIG. 21. The molecular ions observed in both positive and negative ESI-MS [M+1]+1 were m/z 266.10 and [M-1]-1 m/z 264.10, respectively and these peaks are consistent with the expected mass of the negative molecular ions of tizoxanide alkoxide.

### 1.2.6. FT-IR

Fourier Transform Infrared Spectroscopy (FTIR) is an analytical technique, which may be valuable for characterization of an organic molecule. In FTIR, the absorption of infrared energy by the vibrational modes associated to the stretching and bending of specific bonds in a molecule is recorded. The resulting spectra is a combination of the distinctive absorption characteristics of the bonds and functional groups in the molecule. Thus, the FTIR spectra becomes a “fingerprint” of the molecule for identification or comparison against a known spectra. In the case of RM-5071, the spectrum can be compared against the spectrum of desacetyl-NTZA.

### 1.2.6.1 Materials and Equipment

The equipment used by the MCC to perform the FT-IR analysis of RM-5071 and desacetyl-NTZA is the Thermo iS50 spectrometer equipped with a Continuum IR microscope. The FTIR equipment used in Landen, Belgium for FTIR analysis is a Shimadzu IRafinity fitted with Attenuated Total Reflectance accessory.

### 1.2.6.2. Procedure

The samples were isolated by using a spatula. Once isolated, the RM-5071 and desacetyl NTZA samples were mounted (individually) on two diamond microscopy cells and compressed to obtain a thin film. The microscopy films were put on the stage of a Continuum IR Microscope.

### 1.2.6.3. Results

The FTIR technique is usually used to obtain a spectral “fingerprint” of the sample for identification or comparison with the spectrum of a known compound or a compound from a computer database search. The FTIR spectra for RM-5071 and desacetyl-NTZA is in Error! Reference source not found.. A summary of the identified functional groups is for both samples are presented in Table 19. Error! Reference source not found. contains the FTIR spectra obtained in Landen, Belgium.

TABLE 19
Summary of FTIR Results.

| | | Wavenumber |
|---|---|---|
| | | region |
| Sample | Functionality | (cm⁻¹) |
| RM-5071 | C—H | 3058 |
| | Ring Stretch | 1510-1422 |
| | C—O | 1261-1007 |
| | C═C | 739 |
| Desacetyl NTZA | N—H | 3259 |
| | C—H | 3114, 3071 |
| | C═O | 1672 |
| | C═C | 1607, 898 |
| | NO₂ | 1538 |
| | Ring Stretch | 1477 |
| | C—O | 1266-1039 |

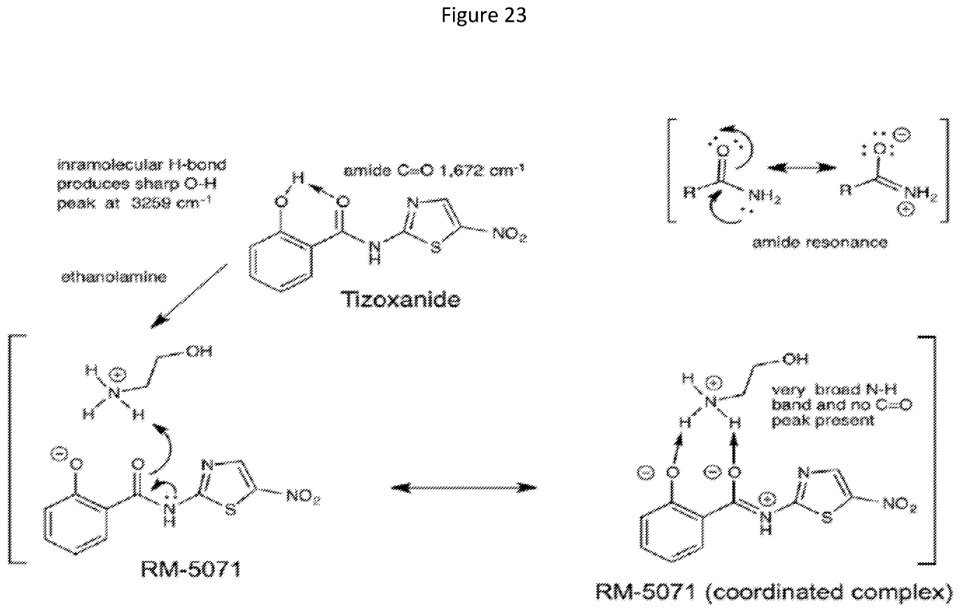

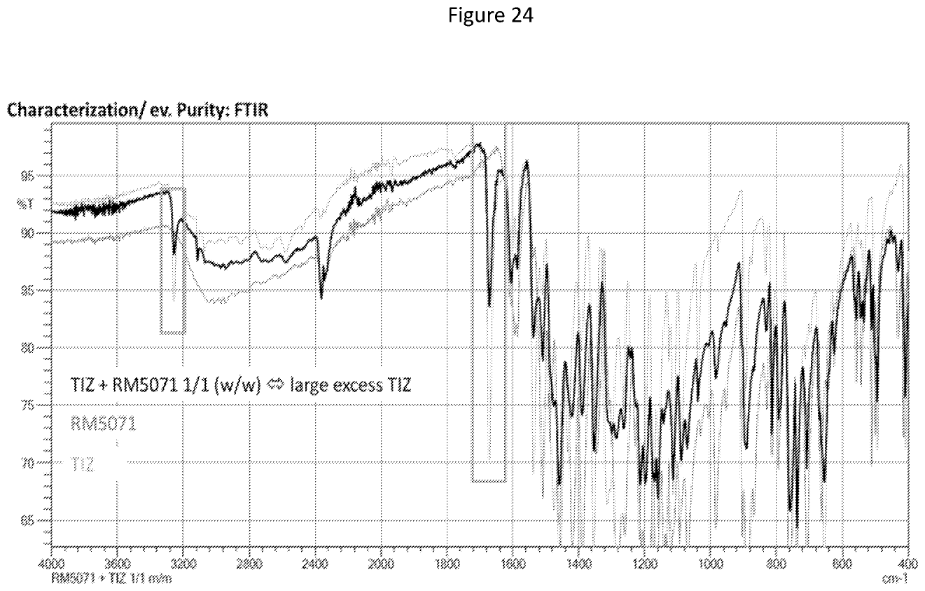

A glance at the overlay of the RM4-5071 and desacetyl-NTZA FTIR spectra shows similarities and striking differences that can be analyzed to render important information about RM4-5071 salt. The differences in the spectra are likely due to the presence of the ethanolammonium ion in RM4-5071 and thus, the different chemical environment surrounding the tizoxanide alkoxide counter ion. Both spectra show a broad peak absorption in the region of 3000 cm⁻¹ typically characteristic of the O-H and N-H stretch modes common to both molecules. The spectrum of desacetyl-NTZA shows the a sharp intense peak at ˜3250cm⁻¹ and a sharp but less intense peak at ˜3100 cm⁻¹ that are not present in the RM-5071 spectrum. In addition, there is a sharp intense band at 1670 cm⁻¹ typically characteristic of the carbonyl stretch mode present in the spectrum of desacetyl-NTZA that completely disappeared in the spectrum of RM-5071. The disappearance of the amide carbonyl peak around 1672 cm⁻¹ and the peaks at 3100 and 3250 cm⁻¹ (possible N-H or OH signals) suggests a strong resonance contribution of the fenolate-ammonium-amide intramolecular coordination complex as shown in FIG. 23.

The “fingerprint” region of spectra below ˜1500cm⁻¹ is different between both molecules which is expected due to the contribution of the functional groups of the ethanolammonioum ion. The disappearance of the amide carbonyl peak was also confirmed by the results on FT-IR experiments in Belgium. The FT-IR analysis was also performed as powder using an attenuated total reflectance (ATR) accessory. The results also showed a clear difference between RM-5071 and desacetyl-NTZA in the carbonyl peak and N-H stretch peaks. In addition, a 1:1 mixture desacetyl-NTZA and ethanolamine analyzed showed the carbonyl peak 1672 cm⁻¹ contrasting with the RM-5071 spectra, thus confirming that the peak disappears only for the salt and not the mixture of the individual molecules. This supports the formation of the salt complex.

### 1.2.7. XTD

X-ray diffraction is based on constructive interference of monochromatic X-rays and a crystalline sample. The interaction of the incident rays with the sample produces constructive interference (and a diffracted ray) when conditions satisfy Bragg's Law (nλ=2d sin θ). This law relates the wavelength of electromagnetic radiation to the diffraction angle and the lattice spacing in a crystalline sample. X-ray crystallography is a tool used for identifying the atomic and molecular structure of a crystal, in which the crystalline atoms cause a beam of incident X-rays to diffract into many specific directions.

### 1.2.7.1. Materials and Equipment

The equipment used by the MCC to perform the analysis is Rigaku SmartLab X-ray Diffractometer.

### 1.2.7.2. Procedure

The XRD analyses were performed on a Rigaku SmartLab X-ray Diffractometer system equipped with a sealed Copper anode tube, a Cu-K beta filter and a D/teX Ultra detector.

A temperature test was performed in order to determine if any physical and crystallinity changes occurred to the sample RM-5071 upon heating.In order to assess if any change occurred two set of experiments were performed as follows:

Experiment A- Two petri dishes were identified as “Fast” and “Slow”. To each petri dish, approximately 2 spatulas of sample RM-5071 were added. Then, the oven was turned on and set to a temperature of-160-165° C. After reaching the temperature, the petri dishes were placed in the oven for 15 minutes. Once the time was completed, the oven was turned off and the “Fast” sample was removed and left to reach room temperature (RT) in the hood. The “Slow” sample was left in the oven to reach RT in a slower manner. The “Slow” sample was removed from the oven, once it reached RT. The samples were analyzed by XRD.

Experiment B- The procedure described above was followed at a temperature range of ˜110-120° C. After reaching the temperature, the petri dishes were placed in the oven during 10 minutes. The removal of samples from the oven was followed as previosly described. Afterwards, the samples were analyzed by XRD.

### 1.2.7.3. Results

By measuring the angles and intensities of these diffracted beams caused by X-ray diffraction, a three-dimensional picture of the density of electrons within the crystal can be produced. The RM4-5071 sample showed a distinguishable XRD pattern with diffraction signals between 2θ angles of 8 to 37°, which suggest that the material exhibits a crystalline form. The desacetyl-NTZA sample showed a distinguishable XRD pattern with diffraction signals between 2θ angles of 6 to 44°, which suggest that the material exhibits a crystalline form. RM-5071 and the desacetyl-NTZA showed different XRD patterns which suggest they exhibit different crystalline forms (Error! Reference source not found.A-B).

The temperature test was performed in order to determine if any physical and crystallinity changes occurred to the RM-5071 upon heating. The temperatures of these heating experiments were chosen based on the TGA transitions identified FIG. 11, Experiment A was heated up to 160-165° C. and Experiment B 110-120° C. The results of this heating experiments are summarized in Table. The results show that in Experiment A, the sample appearance changed from bright yellow to a black powder with beige portions. The XRD pattern of the beige portion resembles the pattern for desacetyl-NTZA. The XRD diffractogram of the black powder did not resemble the difractogram of desacetyl-NTZA nor RM-5071 and was characteristic of an amorphous material. The results of Experiment B show that at 110-120° C. the material does not change significantly as evidenced by the absence of physical changes in the powder and on the XRD pattern before and after heating.

TABLE 20
Summary of XRD-Temperature Test Results

| Experiment | Appearance | XRD Results | |
|---|---|---|---|
| Descriptions | Observations | Summary | Conclusions |
| Experiment A | A large | The black | Amorphous |
| Temp: 160-165° C. | portion of | portion of the | |
| Fast and | both samples | Fast and Slow | |
| slow cooling | (Fast and | samples did | |
| experiment. | Slow) showed | not show | |
| | a black color | diffraction | |
| | (burnt-like) | signals. | |
| | exhibiting a | The beige portion of | Signals |
| | brittle | the Fast and Slow | similar to |
| | consistency. | samples showed | desacetyl- |
| | There were | weak diffraction | NTZA |
| | small portions | signals around | |
| | showing a | 6.6, 13.3, | |
| | beige color | 16.5, 24.5, | |
| | and powder | and 27.5°, | |
| | consistency. | at 2θ | |
| Experiment B | No physical | The Fast and | Same |
| Temp: 110-120° C. | changes were | Slow showed | diffraction |
| Fast and | noticed. | main diffraction | pattern as |
| slow cooling | | signals around | RM-5071 |
| experiment. | | 8.5, 11.2, | |
| | | 16.8, 19.5, | |
| | | 20.9, 25.6, | |
| | | 27.0 and 36.1°, | |
| | | at 2θ. | |

### 2. Conclusion

This report discussed the chemical and physical characterization data compiled for RM-5071. In addition, the chemical and physical properties of RM-5071 were compared with those of its precursor desacetyl-NTZA.

RM-5071 is a yellow powder with a mean particle size around 11 μm as determined by laser diffraction analysis and confirmed by scanning electron microscopy images. It forms agglomerates that can be dispersed by mechanical action, sonication, or air pressure in a venturi dispersor. The melting point ranges from 146 to 148° C. The thermal properties of the material studied by thermogravimetric analysis and differential scanning calorimetry show that the solid is stable up to 163° C. at which it undergoes an irreversible transition. This temperature is lower than the first transition temperature of desacetyl-NTZA. The XRD reflect that RM-5071 has crystalline structure yet different from desacetyl-NTZA.

RM-5071 is very soluble on DMF, DMSO, ACN:Water, and less than slightly soluble in water as defined by the USP. It is soluble (<0.033 mg/mL) in methanol, ethanol and isopropanol and very soluble in binary mixtures 25:75 and 50:50 of water: ethanol or isopropanol. The pKa of the phenolic proton of RM-5071 is 8.8 as determined by acid base titration. The UV-Vis characterization showed peaks on the visible region on the range 409-431 nm for all solvents. The electrospray mass spectrometry results show evidence of the presence of desacetyl-NTZA in solution most easily detected in negative mode and after chromatography of the RM-5071 sample in both negative and positive mode confirming the expected mass to charge ratio. The FTIR data may suggest the presence of a proposed equilibrium among tautomers due to the disappearance of the characteristic carbonyl band in the IR spectrum of RM-5071 as compared to the spectra of pure desacetyl-NTZA and of a 1:1 mixture of desacetyl-NTZA with ethanolamine. The FTIR spectra can be used to differentiate RM-5071 from its precursor.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. An amine containing salt of a compound having formula: 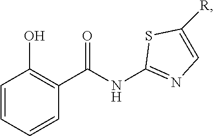
wherein R is NO₂ or a halogen.

2. The amine containing salt of claim 1, wherein R is NO₂.

3. (canceled)

4. The amine containing salt of claim 2, wherein the amine containing salt is an alkyl amine salt, an alkoxy amine salt or a cycloalkyl amine salt.

5. The amine containing salt of claim 2, wherein the amine containing salt is an ethanolamine salt of the compound.

6. The amine containing salt of claim 2, wherein the amine containing salt is a morpholine salt of the compound.

7. The amine containing salt of claim 2, wherein the amine containing salt is a propanolamine salt of the compound.

8. The amine containing salt of claim 2, wherein the amine containing salt is an N-methylpiperazine salt of the compound.

9. (canceled)

10. A batch of the amine containing salt of claim 1 having a purity of at least 90%.

11. (canceled)

12. A pharmaceutical composition comprising the amine containing salt of claim 1 and a pharmaceutically acceptable excipient.

13. (canceled)

14. (canceled)

15. The pharmaceutical composition of claim 12, wherein R is NO₂ and when the composition is administered to a mammal, the composition provides at least one of the following (a) a maximum concentration of the compound in a plasma of the mammal faster than a pharmaceutical composition comprising nitazoxanide, (b) a AUC₀₋₁₂ₕ concentration of the compound in a plasma of the mammal of no less than that of a pharmaceutical composition comprising nitazoxanide, (c) a AUC₀₋₁₂ₕ concentration of the compound and a glucorono form of the compound in a plasma of the mammal of no less than that of a pharmaceutical composition comprising nitazoxanide and (d) a maximum concentration of the compound in a plasma of a mammal in 1 hour or less.

16. (canceled)

17. (canceled)

18. (canceled)

19. (canceled)

20. (canceled)

21. (canceled)

22. The pharmaceutical composition of claim 15, wherein the salt is an ethanolamine salt of tizoxanide.

23. A method of making an amine containing salt of a thiazolide compound, comprising reacting a thiazolide compound of formula 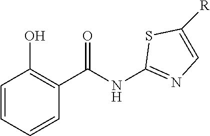
with an amine containing compound to produce an amine containing salt of the thiazolide compound, wherein R is NO₂ or Cl.

24. The method of claim 23, wherein R is NO₂.

25. (canceled)

26. The method of claim 24, wherein the amine containing compound is a liquid amine containing compound.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. The salt of claim 5 having a particle size from about 4 microns to about 40 microns.

40. The salt of claim 5 having a melting temperature from about 146° C. to about 148° C.

41. The salt of claim 5 in a crystalline form.

42. The salt of claim 5 having a differential scanning calorimetry (DSC) curve as in FIG. 12A.

43. The salt of claim 5, having an X-ray powder diffractogram as determined on a diffractometer using Cu-Kβ radiation at a wavelength of 1.39222 Å, wherein the diffractogram has peaks at 8.5° ±0.2°, 11.2° ±0.2°, 16.8° ±0.2°, 19.5° ±0.2°, 20.9° ±0.2°, 25.6° ±0.2°, 27.0° ±0.2° and 36.1° ±0.2° 2θ.

44. (canceled)

45. A batch comprising at least 0.8 kg of the salt of claim 5.

46. (canceled)
